(19) United States
(12) Patent Application Publication
(10) Pub. No.: US 2021/0299134 A1
(43) Pub. Date: Sep. 30, 2021

(54) TREATMENT OF B CELL MALIGNANCIES

(71) Applicant: MEI Pharma, Inc., San Diego, CA (US)

(72) Inventor: Daniel P. GOLD, San Diego, CA (US)

(21) Appl. No.: 17/268,052

(22) PCT Filed: Aug. 13, 2019
(86) PCT No.: PCT/US2019/046411

§ 371 Date: Feb. 11, 2021

Related U.S. Application Data
(60) Provisional application No. 62/836,511, filed on Apr. 19, 2019.
(60) Provisional application No. 62/775,797, filed on Dec. 5, 2018.
(60) Provisional application No. 62/718,929, filed on Aug. 14, 2018.

(51) Int. Cl. A61K 31/5377

(52) U.S. Cl. CPC A61K 31/5377

(57) ABSTRACT

Provided herein are methods of treating cancer using a phosphoinositide-3-kinase (PI3K) inhibitor. In certain embodiments, the cancer is follicular lymphoma (FL). In certain embodiments, the PI3K inhibitor is administered on a continuous dosing schedule (CS). In other embodiments, the PI3K inhibitor is administered on an intermittent dosing schedule (IS).

### CROSS REFERENCE TO RELATED APPLICATIONS

This application claims the benefit of U.S. Provisional Application No. 62/718,929, filed Aug. 14, 2018, U.S. Provisional Application No. 62/775,797, filed Dec. 5, 2018, and U.S. Provisional Application No. 62/836,511, filed Apr. 19, 2019; the disclosure of each of the prior applications is considered part of, and is incorporated by reference in, the disclosure of this application.

### BACKGROUND OF THE DISCLOSURE

Phosphoinositide-3-kinases (PI3Ks) play a variety of roles in normal tissue physiology, with p110α having a specific role in cancer growth, p110β in thrombus formation mediated by integrin α_{Π}β₃, and p110γ, in inflammation, rheumatoid arthritis, and other chronic inflammation states. Inhibitors of PI3K have therapeutic potential in the treatment of various proliferative diseases, including cancer.

### SUMMARY OF THE DISCLOSURE

Some embodiments provided herein describe a method of treating cancer, comprising administering to a subject in need thereof a single pharmaceutical composition consisting of: (i) about 30 mg, about 60 mg, about 120 mg, or about 180 mg of a compound of Formula (I):

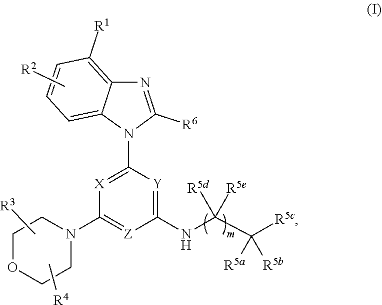

or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
   —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
   —S(O)₂NR^{1b}R^{1c};
R^{5f} and R^{5g} are each independently (a) hydrogen or halo; (b) C₁₋₆ alkyl,
   C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or
   —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h},
   —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl; and
(ii) one or more pharmaceutically acceptable carriers.

In some embodiments of the methods provided herein, about 60 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject daily.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day, twice per day, or three times per day.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day.

In some embodiments of the methods provided herein, about 60 mg/day of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on a 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two 28-day cycles.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for a period of up to about 7 days.

In some embodiments of the methods provided herein, the days over which the compound of Formula (I), or an enantiomer, a mixture of enantiomers, a mixture of two or more diastereomers, or an isotopic variant thereof; or a pharmaceutically acceptable salt, solvate, hydrate, or prodrug thereof are intermittent.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof is administered to the subject for about 7 consecutive days in a 28-day cycle.

In some embodiments of the methods provided herein, the method comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject until disease progression or intolerable toxicity.

In some embodiments of the methods provided herein, the method comprises a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two CS 28-day cycles.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once weekly after the at least two CS 28-day cycles until disease progression or intolerable toxicity.

In some embodiments of the methods provided herein, the method further comprising an IS, comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle after the at least two CS 28-day cycles.

In another aspect provided herein is a method of treating cancer, comprising administering to a subject in need thereof a therapeutically effective amount of a compound of Formula (I):

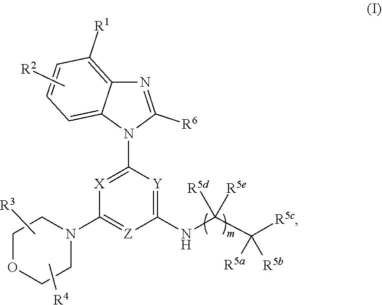

or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
   —S(O)₂NR^{1b}R^{1c}; wherein each R^{1a}, R^{1b}, R^{1c}, and R^{1d} is independently (i) hydrogen;
   (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
   —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or
   —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h},
   —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of about 7 days in a 2 8-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof is administered to the subject for about 7 consecutive days in a 28-day cycle.

In some embodiments of the methods provided herein, the method comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle. In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three 28-day cycles, wherein:
(i) the first two 28-day cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for two 28-day cycles; and
(ii) the third 28-day cycle comprises an intermittent dosing schedule (IS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for only the first 7 consecutive days of the 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three cycles, wherein:
(i) the first two cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for two cycles; and
(ii) the subsequent cycle(s) comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for only the first 7 consecutive days in each subsequent cycle.

In an aspect provided herein is a method of treating cancer, comprising administering to a subject in need thereof a therapeutically effective amount of a compound of Formula (I):

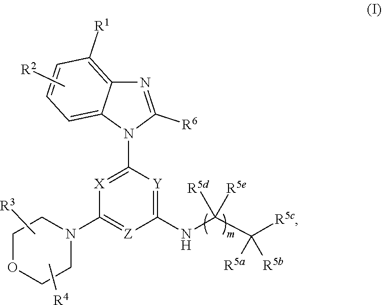

or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
   —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or
   —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a}; wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl, wherein the method comprises at least three 28-day cycles,
wherein:
   (i) the first two cycles comprise a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle; and
   (ii) the third and subsequent cycles comprise an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle. In some embodiments of the methods provided herein, the IS is continued until progression of disease. In some embodiments of the methods provided herein, progression of disease is observed, the subject resumes CS. In some embodiments of the methods provided herein, the CS is continued until unacceptable toxicity.

In some embodiments of the methods provided herein, T-cells are recovered and/or re-populated during the 21 days without treatment.

In some embodiments of the methods provided herein, regulatory T-cells (TREG) and/or effector T-cells are recovered and/or re-populated during the 21 days without treatment.

In some embodiments of the methods provided herein, the incidence of at least one toxicity is reduced.

In some embodiments of the methods provided herein, the at least one toxicity is enterocolitis, a cutaneous toxicity, liver toxicity, pulmonary toxicity, infection, or any combination thereof.

In some embodiments of the methods provided herein, about 60 mg of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease progression occurs.

In some or additional embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered daily to the subject on a continuous dosing schedule (CS) after disease progression occurs on an intermittent dosing schedule (IS).

In some embodiments of the methods provided herein, the cancer is acute leukemia, acute lymphocytic leukemia, acute myelocytic leukemia, myelodysplastic syndrome, refractory anemia, refractory anemia with ringed sideroblasts, refractory anemia with excess blasts, refractory anemia with excess blasts in transformation, preleukemia, chronic myelomonocytic leukemia, chronic myelocytic (granulocytic) leukemia, chronic lymphocytic leukemia, hairy cell leukemia; polycythemia vera, Hodgkin's disease, non-Hodgkin's disease, multiple myeloma, Waldenstrom's macroglobulinemia; monoclonal gammopathy of undetermined significance, benign monoclonal gammopathy, heavy chain disease, bone and connective tissue sarcoma, brain tumor, breast cancer, adrenal cancer, thyroid cancer, pancreatic cancer, pituitary cancer, eye cancer, vaginal cancer, vulvar cancer, cervical cancer, uterine cancer, ovarian cancer, esophageal cancer, stomach cancer, colon cancer, rectal cancer, liver cancer, hepatocellular carcinoma, hepatoblastoma, gallbladder cancer, adenocarcinoma, cholangiocarcinoma, lung cancer, testicular cancer, prostate cancer, penal cancer; oral cancer, basal cancer, salivary gland cancer, pharynx cancer, skin cancer, kidney cancer, bladder cancer, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinoma.

In some embodiments of the methods provided herein, the cancer is leukemia, lymphoma, multiple myeloma, sarcoma, a brain tumor, breast cancer, adrenal cancer, thyroid cancer, pancreatic cancer, pituitary cancer, cervical cancer, ovarian cancer, esophageal cancer, stomach cancer, colon cancer, rectal cancer, liver cancer, lung cancer, testicular cancer, prostate cancer, or skin cancer.

In some embodiments of the methods provided herein, the cancer is the cancer is acute leukemia, acute lymphocytic leukemia, acute myelocytic leukemia, chronic myelomonocytic leukemia (CMML), chronic myelocytic (granulocytic) leukemia, chronic lymphocytic leukemia, hairy cell leukemia, Hodgkin's disease, non-Hodgkin's disease, smoldering multiple myeloma, nonsecretory myeloma, osteosclerotic myeloma, plasma cell leukemia, solitary plasmacytoma, extramedullary plasmacytoma, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, diffuse malignant lymphoma, non-small cell lung cancer, large-cell carcinoma, small-cell lung cancer, or basal cell carcinoma.

In some embodiments of the methods provided herein, the cancer is chronic lymphocytic leukemia or non-Hodgkin's lymphoma.

In some embodiments of the methods provided herein, the cancer is a hematological cancer or malignancy.

In some embodiments of the methods provided herein, the cancer is a B-cell malignancy.

In some embodiments of the methods provided herein, the cancer is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments of the methods provided herein, the cancer is non-Hodgkin's lymphoma diffuse large B-cell lymphoma (DLBCL).

In some embodiments of the methods provided herein, the cancer is relapsed/refractory diffuse large B-cell lymphoma (r/r DLBCL).

In some embodiments of the methods provided herein, the diffuse large B-cell lymphoma is of the activated B-cell (ABC DLBCL) or Germinal center B-cell (GCB DLBCL).

In some embodiments of the methods provided herein, the cancer is follicular lymphoma (FL). In some embodiments of the methods provided herein, the FL is relapsed/refractory FL. In some embodiments of the methods provided herein, the FL is relapsed/refractory FL after failure of at least two prior lines of systemic therapy in the subject. In some embodiments of the methods provided herein, the FL is relapsed/refractory FL after failure of at least two prior lines of systemic therapy in the subject, wherein the systemic therapy comprises an antiCD20 antibody and/or chemotherapy with an alkylating agent or a purine analog.

In some embodiments provided herein are methods of treating follicular lymphoma (FL) in a subject in need thereof, wherein the subject has failed two or more prior chemotherapies. In some embodiments provided herein are methods of treating follicular lymphoma (FL) in a subject in need thereof, wherein the subject has failed two or more prior systemic chemotherapies. In some embodiments provided herein are methods of treating follicular lymphoma (FL) in a subject in need thereof, wherein the subject has failed two or more prior systemic chemotherapies, wherein each systemic chemotherapy is selected from the group consisting of an antiCD20 antibody, an alkylating chemotherapeutic agent, and a chemotherapeutic purine analog.

In another aspect herein is provided a method of treating follicular lymphoma (FL), comprising administering to a subject in need thereof a single pharmaceutical composition consisting of: (i) a compound of Formula (I):

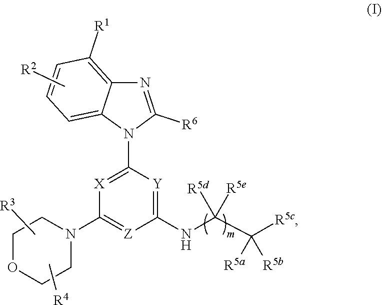

or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a}; wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl.

In some embodiments of the methods provided herein, R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments of the methods provided herein, R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments of the methods provided herein, wherein R^{5a} and R^{5b} are each methyl, optionally substituted with one, two, or three halo(s).

In some embodiments of the methods provided herein, nisi.

In some embodiments of the methods provided herein, R^{5f} and R^{5g} are each hydrogen.

In some embodiments of the methods provided herein, n is 0.

In some embodiments of the methods provided herein, m is 0.

In some embodiments of the methods provided herein, the compound of Formula (I) is of Formula (XI):

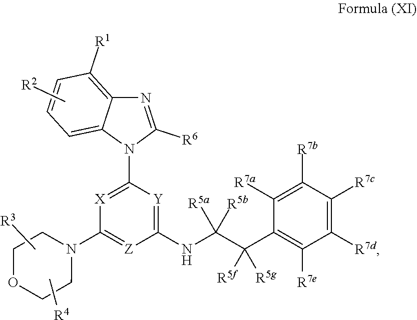

or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or
two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

In some embodiments of the methods provided herein:
X, Y, and Z are each N;
R¹ and R² are each hydrogen;
R³ and R⁴ are each hydrogen;
R^{5a} is C₁₋₆ alkyl;
R^{5b} is C₁₋₆ alkyl;
R^{5c} is —(CH₂)-phenyl, wherein R^{5c} is optionally substituted with one, two, three, or four substituents Q;
R^{5d} and R^{5e} are each hydrogen;
R⁶ is CHF₂;
m is 0; and
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

In some embodiments of the methods provided herein, R^{5a} and R^{5b} are each methyl, optionally substituted with one or more halos.

In some embodiments of the methods provided herein, the compound of Formula (I) is Compound A35:

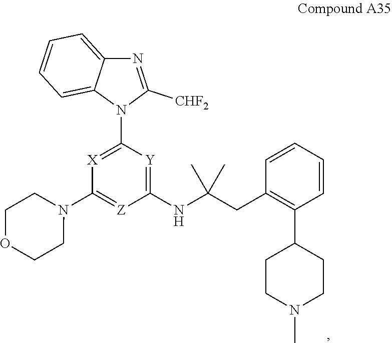

or an isotopic variant thereof, a pharmaceutically acceptable salt, solvate, hydrate, or prodrug thereof.

In some embodiments of the methods provided herein, the compound of Formula (I) is Compound A36:

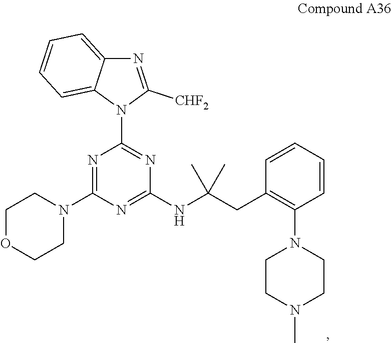 [SMILES: CN1CCN(c2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A68:

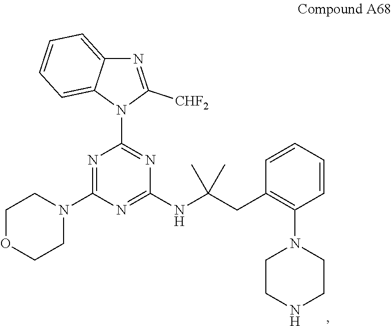 [SMILES: CC(C)(Cc1ccccc1N1CCNCC1)Nc1nc(N2CCOCC2)nc(-n2c(C(F)F)nc3ccccc32)n1]

or an isotopic variant thereof, a pharmaceutically acceptable salt, solvate, hydrate, or prodrug thereof.

In some embodiments of the methods provided herein, the compound of Formula (I) is Compound A70:

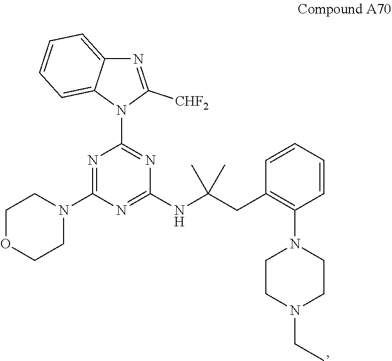 [SMILES: CCN1CCN(c2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A37:

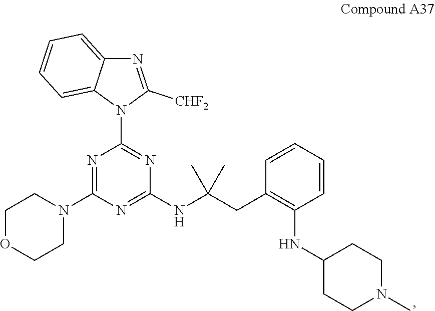 [SMILES: CN1CCC(Nc2ccccc2CC(C)(C)Nc2nc(N3CCOCC3)nc(-n3c(C(F)F)nc4ccccc43)n2)CC1]

or an isotopic variant thereof, a pharmaceutically acceptable salt, solvate, hydrate, or prodrug thereof.

In some embodiments of the methods provided herein, the compound of Formula (I) is Compound A38:

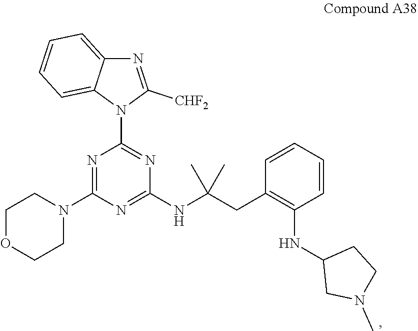 [SMILES: CN1CCC(Nc2ccccc2CC(C)(C)Nc2nc(N3CCOCC3)nc(-n3c(C(F)F)nc4ccccc43)n2)C1]

or an isotopic variant thereof, a pharmaceutically acceptable salt, solvate, hydrate, or prodrug thereof.

In some embodiments of the methods provided herein, the compound of Formula (I) is Compound A41:

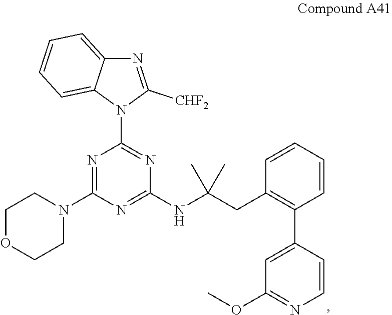 [SMILES: COc1cc(-c2ccccc2CC(C)(C)Nc2nc(N3CCOCC3)nc(-n3c(C(F)F)nc4ccccc43)n2)ccn1]

or an isotopic variant thereof, a pharmaceutically acceptable salt, solvate, hydrate, or prodrug thereof.

In some embodiments of the methods provided herein, the compound of Formula (I) is Compound A42:

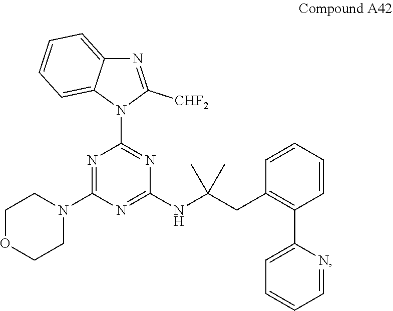 [SMILES: CC(C)(Cc1ccccc1-c1ccccn1)Nc1nc(N2CCOCC2)nc(-n2c(C(F)F)nc3ccccc32)n1]

or an isotopic variant thereof, a pharmaceutically acceptable salt, solvate, hydrate, or prodrug thereof.

In some embodiments of the methods provided herein, the compound of Formula (I) is Compound A43:

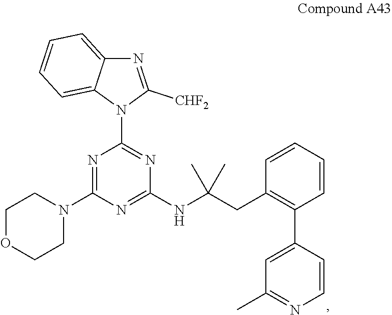 [SMILES: Cc1cc(-c2ccccc2CC(C)(C)Nc2nc(N3CCOCC3)nc(-n3c(C(F)F)nc4ccccc43)n2)ccn1]

or an isotopic variant thereof, a pharmaceutically acceptable salt, solvate, hydrate, or prodrug thereof.

In some embodiments of the methods provided herein, the compound of Formula (I) is Compound A44:

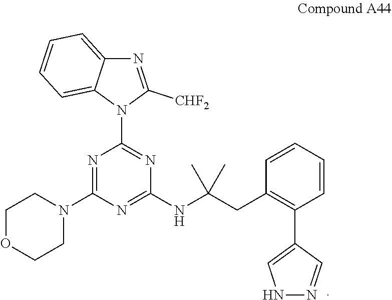 [SMILES: CC(C)(Cc1ccccc1-c1cn[nH]c1)Nc1nc(N2CCOCC2)nc(-n2c(C(F)F)nc3ccccc32)n1]

or an isotopic variant thereof, a pharmaceutically acceptable salt, solvate, hydrate, or prodrug thereof.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject orally.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A-1B Schematic representations of dosing schedules: A depicts the Continuous Schedule (CS); and B depicts the Intermittent Schedule (IS).

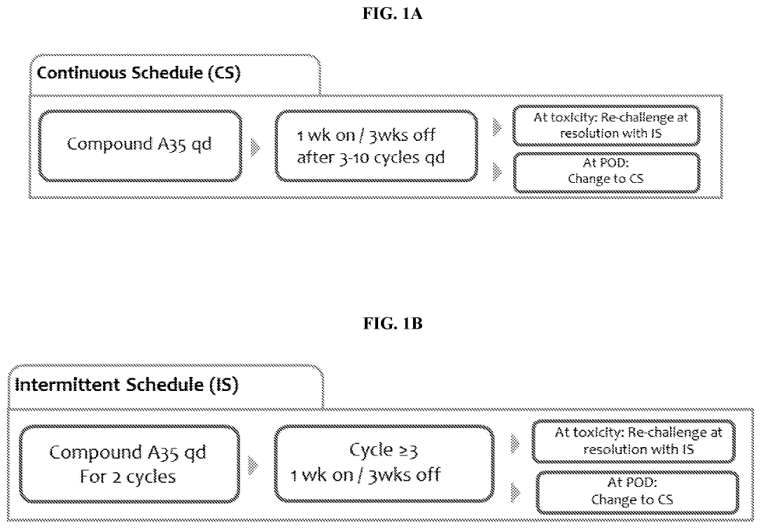

FIG. 2. Graphical representation showing the best change from baseline of measurable lesions for monotherapy with Compound A35 on a continuous dosing schedule (CS) or intermittent dosing schedule (IS) in follicular lymphoma patients.

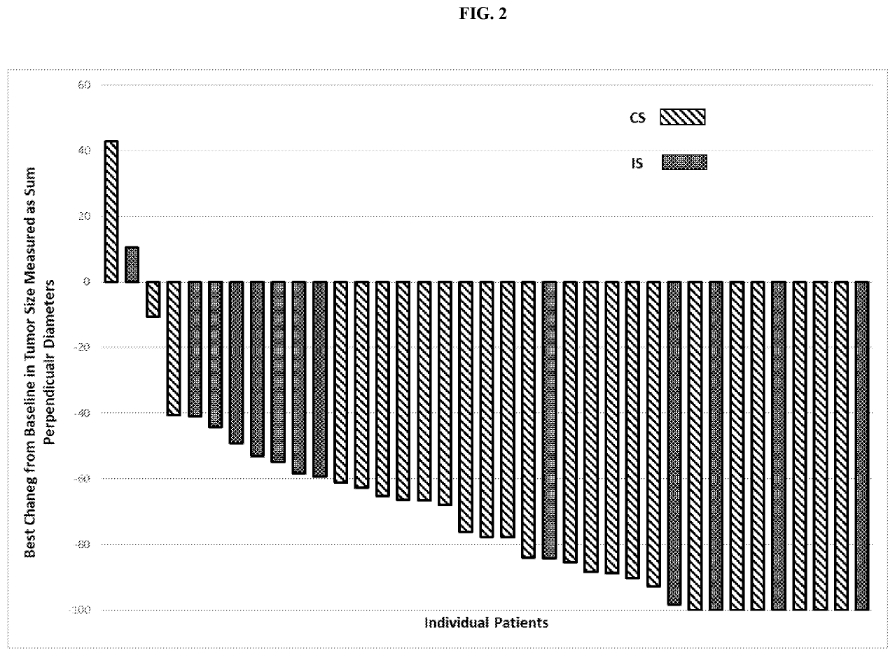

FIG. 3. Graphical representation showing preferential tumor exposure of Compound A35 over a 4-hour and 24-hour period.

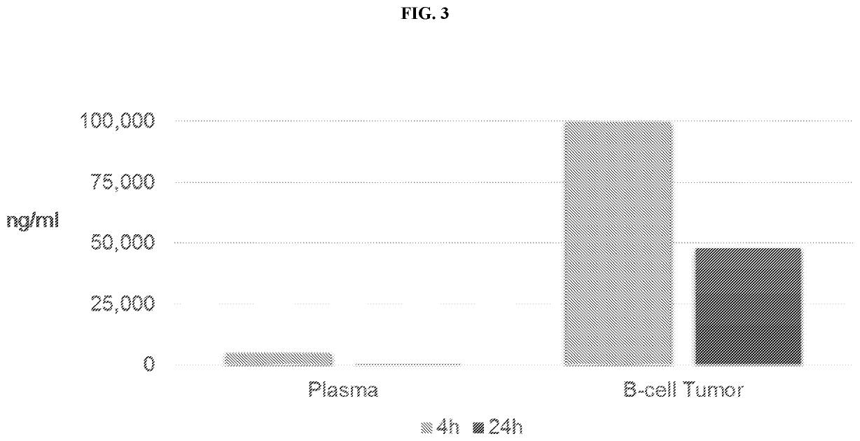

FIG. 4. Graphical representation showing preferential retention of Compound A35 compared to idelalisib in murine B-cell tumors.

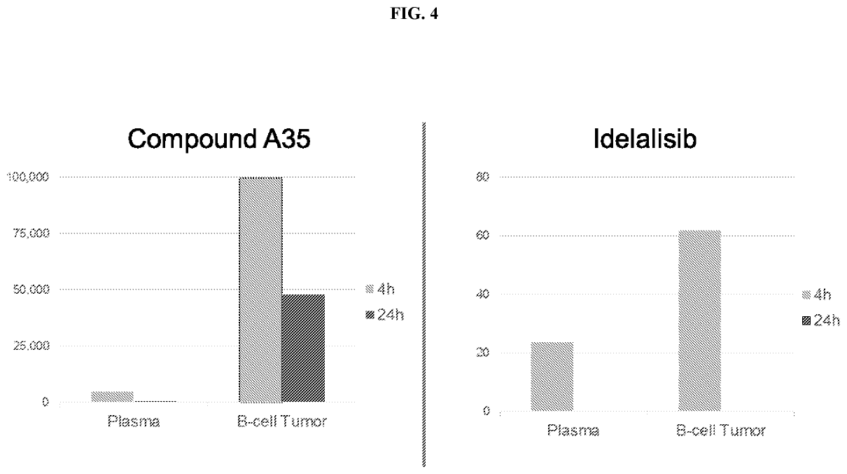

FIG. 5 Schematic representation of monotherapy treatment paradigm with Compound A35 in patients with R/R FL.

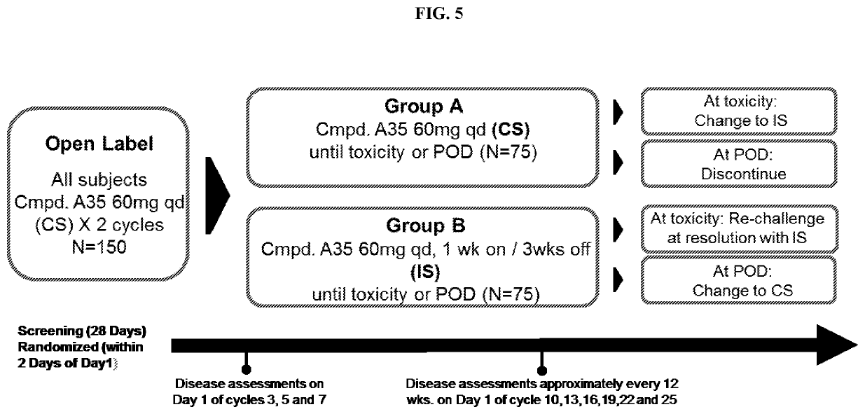

FIGS. 6A-6B. Graphical representation of Intermittent Dosing Schedule with Compound A35 (A) compared to parsaclisib (B) to maintain disease control.

### DETAILED DESCRIPTION OF THE INVENTION

Some embodiments provided herein describe pharmaceutical compositions comprising a PI3K delta inhibitor and methods for treating patients with B cell malignancies with a PI3K inhbitor. In some embodiments, the dosing regimens and schedules described herein reduce toxicities associate with PI3K delta inhibitors.

Class I phosphatidylinositol 3-kinases (PI3Ks) regulate numerous cellular functions. PI3Ks are composed of regulatory (p85) and catalytic (p110) subunits, with the catalytic unit consisting of 4 distinct isoforms designated α, β, γ, and δ. PI3K5 is primarily expressed in lymphocytes where it plays a key role in normal lymphocyte biology including proliferation, homing and survival. PI3K5 is frequently active in B-cell malignancies and is central to multiple B-cell receptor (BCR) signaling pathways that drive proliferation, survival, homing and retention of malignant B-cells in lymphoid tissue and bone marrow. Small molecule PI3K delta (or PI3K5) inhibitors are effective for the treatment of B-cell malignancies, including chronic lymphocytic leukemia (CLL), follicular lymphoma and other B-cell lymphomas. However, in some instances, toxicities associated with the PI3K5 are severe and have been fatal in some patients. Toxicities reported with PI3K5 inhibitors (e.g., idelalisib, parsaclisib (INCB050465), copanlisib, duvelisib, umbralisib, etc.) include but are not limited to enterocolitis (manifested as diarrhea/colitis), cutaneous toxicities (e.g., rashes), liver toxicity (manifested as elevation of transaminases), pulmonary toxicity (manifested as non-infectious pneumonitis), and infections. These toxicities may be severe and have been fatal in some patients. The frequency, severity and time to onset of these adverse events (AEs) vary among PI3K5 inhibitors. Enterocolitis, rash, and transaminitis have been reported in certain clinical studies of PI3K5 in patients with B-cell malignancies. In certain instances, lymphocytic infiltrates have reported in biopsies obtained from subjects with colitis and/or severe skin rash with corticosteroid therapy being an effective treatment approach in patients who developed diarrhea and rash.

A better understanding of the pathogenesis of these toxicities may help in developing approaches to mitigate their risk. Several lines of investigations have suggested that some of these toxicities are related to dysfunction in immune homeostasis. An immune mechanism for PI3K5-associated enterocolitis has been hypothesized based on observations that include mice with genetic inactivation of p110δ develop an autoimmune-like colitis; histopathologic data from patients with diarrhea/colitis associated with PI3K5 inhibitors show an intraepithelial lymphocytosis, indicative of an immune reaction; and some patients with late onset PI3K5 inhibitors-associated diarrhea/colitis do not respond to antidiarrheal or empiric antimicrobial therapy but may respond to treatment with corticosteroids, supporting an immune mechanism for the diarrhea.

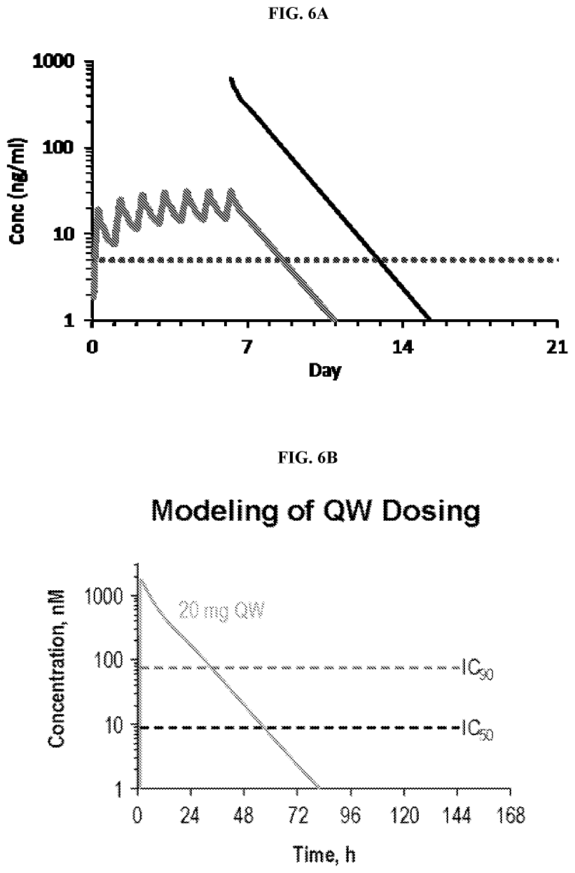

There is also evidence pointing to a role for the PI3K pathway in T lymphocytes, which may explain these immune dysregulations. For example, in mice, genetic inactivation of p110δ results in a decrease of the function of regulatory T-cells (TREGs), a subset population of T-cells. TREGs have been shown to have an important role in controlling auto-immunity. In mice, p110δ was shown to be required for mounting and effective T-cell responses to viral and bacterial infections. In some instances, PI3K5 inhibition results in various immune-mediated toxicities such as enterocolitis and skin toxicity due to TREG suppression, as well as infections due to suppression of B-cells and effector T-cells. In some instances, treatment regimens with small molecule PI3K5 delta (or PI3K5) inhibitors on an intermittent dosing schedule (IS) are used. However, in certain instances, progression of disease is observed in subjects who are treated for B-cell malignancies, including chronic lymphocytic leukemia (CLL) and lymphomas with small molecule PI3K5 inhibitors (e.g., parsaclisib (INCB050465)) on an IS dosing regimen (see FIG. 6B). It has been demonstrated that parsaclisib once weekly dosing resulted in plasma levels >IC₉₀ for 1.5/7 days (i.e., 32%). For parsaclisib, plasma approximates tissue levels and off target ˜5 days of 7 was not sufficient to hold response to treatment in most patients. In some embodiments, the methods of treatment and dosing regimens and schedules described herein provide an efficacious and tolerable treatment of cancer. In some embodiments, the methods of treatment and dosing regimens and schedules described herein improve the frequency, severity and time to onset of the adverse events (AEs) associated with PI3K delta inhibitors. In some embodiments, the methods of treatment and dosing regimens and schedules described herein, including IS dosing regimens, result in partial or complete remission. In some embodiments, the methods of treatment and dosing regimens and schedules described herein, including IS dosing regimens (e.g., one week on/three week off dosing), result in plasma levels of >IC₉₀ for 9/28 days (i.e., 32%) for the compounds described herein. For the compounds described herein (e.g., Compound A35), plasma levels underestimate tissue levels, and higher levels of the compounds described herein are predicted in tumor versus plasma (FIG. 6A).

### Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “therapeutically effective amount” or “effective amount” are meant to include the amount of a compound that, when administered, is sufficient to prevent development of, or alleviate to some extent, one or more of the symptoms of the disorder, disease, or condition being treated. The terms “therapeutically effective amount” or “effective amount” also refer to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition.

The term “naturally occurring” or “native” when used in connection with biological materials such as nucleic acid molecules, polypeptides, host cells, and the like, refers to materials which are found in nature and are not manipulated by man. Similarly, “non-naturally occurring” or “non-native” refers to a material that is not found in nature or that has been structurally modified or synthesized by man.

The term “PI3K” refers to a phosphoinositide 3-kinase or variant thereof, which is capable of phosphorylating the inositol ring of PI in the D-3 position. The term “PI3K variant” is intended to include proteins substantially homologous to a native PI3K, i.e., proteins having one or more naturally or non-naturally occurring amino acid deletions, insertions, or substitutions (e.g., PI3K derivatives, homologs, and fragments), as compared to the amino acid sequence of a native PI3K. The amino acid sequence of a PI3K variant is at least about 80% identical, at least about 90% identical, or at least about 95% identical to a native PI3K. Examples of PI3K include, but are not limited to, p110α, p110β, p110δ, p110γ, PI3K-C2α, PI3K-C2β, PI3K-C2γ, Vps34, mTOR, ATM, ATR, and DNA-PK. See. Fry, Biochem. Biophys. Acta 1994, 1226, 237-268; Vanhaesebroeck and Waterfield, Exp. Cell. Res. 1999, 253, 239-254; and Fry, Breast Cancer Res. 2001, 3, 304-312. PI3Ks are classified into at least four classes. Class I includes p110α, p110β, p110δ, and p110γ. Class II includes PI3K-C2α, PI3K-C2β, and PI3K-C2γ. Class III includes Vps34. Class IV includes mTOR, ATM, ATR, and DNA-PK. In certain embodiments, the PI3K is a Class I kinase. In certain embodiments, the PI3K is p110α, p110β, p110δ, or p110γ. In certain embodiments, the PI3K is PI3K delta. In certain embodiments, the PI3K is a variant of a Class I kinase. In certain embodiments, the PI3K is a p110α mutant. Examples of p110α mutants include, but are not limited to, R38H, G106V, K111N, K227E, N345K, C420R, P539R, E542K, E545A, E545G, E545K, Q546K, Q546P, E453Q, H710P, I800F, T1025S, M1043I, M1043V, H1047F, H1047R, andH1047Y (Ikenoue et al., Cancer Res. 2005, 65, 4562-4567; Gymnopoulos et al., Proc. Natl. Acad Sci., 2007, 104, 5569-5574).

In certain embodiments, the PI3K is a Class II kinase. In certain embodiments, the PI3K is PI3K-C2α, PI3K-C2p, or PI3K-C2γ. In certain embodiments, the PI3K is a Class III kinase. In certain embodiments, the PI3K is Vps34. In certain embodiments, the PI3K is a Class IV kinase. In certain embodiments, the PI3K is mTOR, ATM, ATR, or DNA-PK.

The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (H), carbon-11 (C), carbon-14 (C), nitrogen-13 (N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a compound contains unnatural proportions of deuterium (D).

The term “alkyl” refers to a linear or branched saturated monovalent hydrocarbon radical, wherein the alkylene may optionally be substituted with one or more substituents Q as described herein. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl. isopropyl, butyl (including all isomeric forms), n-butyl. isobutyl, sec-butyl, n-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “alkylene” refers to a linear or branched saturated divalent hydrocarbon radical, wherein the alkylene may optionally be substituted with one or more substituents Q as described herein. The term “alkylene” encompasses both linear and branched alkylene, unless otherwise specified. In certain embodiments, the alkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene. isobutylene, t-butylene, pentylene (including all isomeric forms), and hexylene (including all isomeric forms). For example, C₁₋₆ alkylene refers to a linear saturated divalent hydrocarbon radical of 1 to 6 carbon atoms or a branched saturated divalent hydrocarbon radical of 3 to 6 carbon atoms.

The term “heteroalkylene” refers to a linear or branched saturated divalent hydrocarbon radical that contains one or more heteroatoms each independently selected from O, S, and N in the hydrocarbon chain. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —CH₂OCH₂—, —CH₂CH₂O—, —CH₂NH—, —CH₂NHCH₂—, —CH₂CH₂NH—, —CH₂S—, —CH₂SCH₂—, and —CH₂CH₂S—. In certain embodiments, heteroalkylene may also be optionally substituted with one or more substituents Q as described herein.

The term “alkenyl” refers to a linear or branched monovalent hydrocarbon radical, which contains one or more, in one embodiment, one, two, three, four, or five, in another embodiment, one, carbon-carbon double bond(s). The alkenyl may be optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkenylene” refers to a linear or branched divalent hydrocarbon radical, which contains one or more, in one embodiment, one, two, three, four, or five, in another embodiment, one, carbon-carbon double bond(s). The alkenylene may be optionally substituted with one or more substituents Q as described herein. Similarly, the term “alkenylene” also embraces radicals having “cA” and “Irans” configurations, or alternatively, “E” and “Z” configurations. As used herein, the term “alkenylene” encompasses both linear and branched alkenylene, unless otherwise specified. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, allylene, propenylene, butenylene, and 4-methylbutenylene.

The term “heteroalkenylene” refers to a linear or branched divalent hydrocarbon radical, which contains one or more, in one embodiment, one, two, three, four, or five, in another embodiment, one, carbon-carbon double bond(s), and which contains one or more heteroatoms each independently selected from O, S, and N in the hydrocarbon chain. The heteroalkenylene may be optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, four, or five, in another embodiment, one, carbon-carbon triple bond(s). The alkynyl may be optionally substituted with one or more substituents Q as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C═CH) and propargyl (—CH₂C═CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “cycloalkyl” refers to a cyclic saturated bridged and/or non-bridged monovalent hydrocarbon radical, which may be optionally substituted with one or more substituents Q as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

The term “cycloalkenyl” refers to a cyclic unsaturated, nonaromatic bridged and/or non-bridged monovalent hydrocarbon radical, which may be optionally substituted with one or more substituents Q as described herein. In certain embodiments, the cycloalkenyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclobutenyl, cyclopentenyl, cyclohexenyl, or cycloheptenyl,

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may be optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 2-phenylethyl, and 3-phenylpropyl. In certain embodiments, the aralkyl are optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms independently selected from O, S, N, and P in the ring. A heteroaryl group is bonded to the rest of a molecule through its aromatic ring. Each ring of a heteroaryl group can contain one or two O atoms, one or two S atoms, one to four N atoms, and/or one or two P atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, the heteroaryl may also be optionally substituted with one or more substituents Q as described herein as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms independently selected from O, S, N, and P; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. A heterocyclyl group is bonded to the rest of a molecule through its non-aromatic ring. In certain embodiments, the heterocyclyl is a monocyclic, bicyclic, tricyclic, or tetracyclic ring system, which may be spiro, fused, or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocyclyl may also be optionally substituted with one or more substituents Q as described herein.

The terms “halogen,” “halide,” or “halo” refer to fluorine, chlorine, bromine, and/or iodine.

The term “optionally substituted” is intended to mean that a group or substituent, such as an alkyl, alkylene, heteroalkylene, alkenyl, alkenylene, heteroalkenylene, alkynyl, cycloalkyl, cycloalkenyl, aryl, aralkyl, heteroaryl, heteroaryl-C₁₋₆ alkyl, and heterocyclyl group, may be substituted with one or more substituents Q, each of which is independently selected from, e.g., (a) oxo (═O), halo, cyano (—CN), and nitro (—NO₂); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, four, or five, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)R^{a}R^{d}, —P(O)(OR^{a})R^{d}, —P(O)(OR^{a})(OR^{d}), —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four substituents Q^{a}; or (iii) R^{b} and R^{c} together with the N atom to which they are attached form heteroaryl or heterocyclyl, optionally substituted with one or more, in one embodiment, one, two, three, or four substituents Q^{a}. As used herein, all groups that can be substituted are “optionally substituted,” unless otherwise specified.

In one embodiment, each substituent Q^{a} is independently selected from the group consisting of (a) oxo, cyano, halo, and nitro; and (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —P(O)R^{e}R^{h}, —P(O)(OR^{e})R^{h}, —P(O)(OR^{e})(OR^{h}), —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) R^{f} and R^{g} together with the N atom to which they are attached form heteroaryl or heterocyclyl.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, Rand S.

The phrase “an enantiomer, a mixture of enantiomers, a mixture of two or more diastereomers, or an isotopic variant thereof; or a pharmaceutically acceptable salt, solvate, hydrate, or prodrug thereof” has the same meaning as the phrase “an enantiomer, a mixture of enantiomers, a mixture of two or more diastereomers, or an isotopic variant of the compound referenced therein; or a pharmaceutically acceptable salt, solvate, hydrate, or prodrug of the compound referenced therein; or a pharmaceutically acceptable salt, solvate, hydrate, or prodrug of an enantiomer, a mixture of enantiomers, a mixture of two or more diastereomers, or an isotopic variant of the compound referenced therein.”

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol. isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The terms “resistent,” “relapsed,” or “refractory” refer to a cancer that has a reduced responsiveness to a treatment, e.g., the point at which the cancer does not respond to attempted forms of treatment. The cancer can be resistant at the beginning of treatment or it may become resistant during treatment. The term “refractory” can refer to a cancer for which treatment (e.g., chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

The terms “intermittent dosing schedule” or “IS” refer to drugs (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) dosed or administered less than once daily. In some embodiments herein, IS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof), to a subject once daily for a period of about 7 days in a 28-day cycle. In other embodiments herein, IS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) daily for up to three (e.g., two) 28-day cycles and, in the third cycle and subsequent cycles, dosing or administration of the drug to the subject once daily for a period of about 7 days in a 28-day cycle. In some embodiments, IS is continued until progression of disease occurs/is observed or until an incidence of at least one toxicity is reduced.

The terms “continuous dosing schedule” or “CS” refer to drugs (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) dosed or administered once daily. In some embodiments herein, CS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof), to a subject daily in a 28-day cycle. In other embodiments herein, CS refers to dosing or administration of a drug (e.g., the compound of Formula (I), or an enantiomer, a mixture of enantiomers, a mixture of two or more diastereomers, or an isotopic variant thereof; or a pharmaceutically acceptable salt, solvate, hydrate, or prodrug thereof) daily for >three 28-day cycles and, in the one or more subsequent cycles, the drug is dosed or administered to the subject once daily for a period of about 7 days in a 28-day cycle (i.e., late switch to IS). In some embodiments, the subject on CS is never switched to IS. In some embodiments, CS is continued until intolerable toxicity occurs/is observed.

“Responsiveness” or to “respond” to treatment, and other forms of this term, as used herein, refer to the reaction of a subject to treatment with a therapeutic, e.g., a PI3K inhibitor, alone or in combination, e.g., monotherapy or combination therapy. Responsiveness to a therapy, e.g., treatment with a PI3K inhibitor alone or in combination, can be evaluated by comparing a subject's response to the therapy using one or more clinical criteria, such as IWCLL 2008 (for CLL) described in, e.g., Hallek, M. et al. (2008) Blood 111 (12): 5446-5456; the Lugano Classification described in, e.g., Cheson, B. D. et al. Journal of Clinical Oncology, 32(27): 3059-3067; and the like. Additional classifications of responsiveness are provided by. These criteria provide a set of published rules that define when cancer patients improve (“respond”), stay the same (“stable”) or worsen (“progression”) during treatments.

For example, a subject having CLL can be determined to be in complete remission (CR) or partial remission (PR). For example, according to IWCLL 2008, a subject is considered to be in CR if at least all of the following criteria as assessed after completion of therapy are met: (i) Peripheral blood lymphocytes (evaluated by blood and different count) below 4×10⁹/L (4000 μi); (ii) no hepatomegaly or splenomegaly by physical examination; (iii) absence of constitutional symptoms; and (iv) blood counts (e.g., neutrophils, platelets, hemoglobin) above the values set forth in Hallek, M. et al. Partial remission (PR) for CLL is defined according to IWCLL 2008 as including one of: (i) a decrease in number of blood lymphocytes by 50% or more from the value before therapy; (ii) a reduction in lymphadenopathy, as detected by CT scan or palpation; or (iii) a reduction in pretreatment enlargement of spleen or liver by 50% or more, as detected by CT scan or palpation; and blood counts (e.g., neutrophils, platelets, hemoglobin) according to the values set forth in Hallek, M. et al. In other embodiments, a subject having CLL is determined to have progressive disease (PD) or stable disease (SD). For example, according to IWCLL 2008, a subject is considered to be in PD during therapy or after therapy if at least one of the following criteria is met: (i) progression on lymphadenopathy; (ii) an increase in pretreatment enlargement of spleen or liver by 50% or more, or de novo appearance of hepatomegaly or splenomegaly; (iii) an increase in the number of blood lymphocytes by 50% or more with at least 5000 B lymphocytes per microliter; (iv) transformation to a more aggressive histology (e.g., Richter syndrome); or (v) occurrence of cytopenia (neutropenia, anemia or thrombocytopenia) attributable to CLL. Stable disease (SD) for CLL is defined according to IWCLL 2008 as a patient who has not achieved CR or a PR, and who has not exhibited progressive disease.

For example, in some embodiments, a subject with CLL responds to treatment with a PI3K inhibitor, alone or in combination, if at least one of the criteria for disease progression according to IWCLL is retarded or reduced, e.g., by about 10%, 20%, 30%, 40%, 50%, 60%, 70%, 80%, 90% or more. In another example, a subject responds to treatment with a PI3K inhibitor, alone or in combination, if the subject experiences a life expectancy extension, e.g., extended by about 5%, 10%, 20%, 30%, 40%, 50% or more beyond the life expectancy predicted if no treatment is administered. In another example, a subject responds to treatment with a PI3K inhibitor, alone or in combination, if the subject has one or more of: an increased progression-free survival, overall survival or increased time to progression (TTP), e.g., as described in Hallek, M. et al.

### PI3K Inhibitors

Some embodiments provided herein describe PI3K inhibitors useful for treating B cell malignanices. In some embodiments, the PI3K inhibitor is selective for PI3K delta. Provided here in some embodiments are PI3K inhibitors of Formula (I):

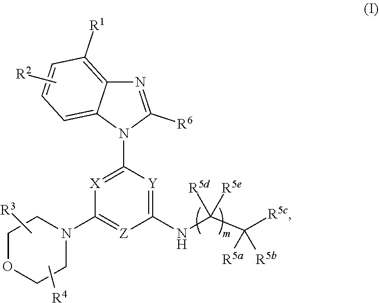

or an enantiomer, a mixture of enantiomers, a mixture of two or more diastereomers, or an isotopic variant thereof; or a pharmaceutically acceptable salt, solvate, hydrate, or prodrug thereof;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, four, or five substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein two substituents Q that are adjacent to each other optionally form a C₃₋₁₀ cycloalkenyl, C₆₋₁₄ aryl, heteroaryl, or heterocyclyl, each optionally substituted with one, two, three, or four substituents Q^{a}.

In some embodiments, the compound of structural Formula (I) is not 4-(2-(difluoromethyl)-1H-benzo[d]imidazol-1-yl)-6-morpholino-N-(2-phenyl-2-(pyrrolidin-1-yl)ethyl)-1,3,5-triazin-2-amine or 6-(2-(difluoromethyl)-1H-benzo[d]imidazol-1-yl)-N-(1-(4-((R)-3-(methoxymethyl)morpholino)phenyl)ethyl)-2-morpholinopyrimidin-4-amine.

In one embodiment of a compound of Formula (I), X, Y, and Z are each independently N or CR^{X}, with the proviso that at least two of X, Y, and Z are nitrogen atoms; where R^{X} is hydrogen or C₁₋₆ alkyl. In another embodiment of a compound of Formula (I), X, Y, and Z are N. In some embodiments, R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments, R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments, R^{5a} and R^{5b} are each methyl, optionally substituted with one or more halo.

In some embodiments, R^{5f} and R^{5g} are each hydrogen.

In some embodiments of compounds of structural Formula (I):
X, Y, and Z are each N;
R¹ and R² are each hydrogen;
R³ and R⁴ are each hydrogen;
R^{5a} is C₁₋₆ alkyl;
R^{5b} is C₁₋₆ alkyl;
R^{5c} is —(CH₂)-phenyl, wherein R^{5c} is optionally substituted with one, two, three, or four substituents Q;
R^{5d} and R^{5e} are each hydrogen;
R⁶ is CHF₂; and
m is 0;
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
   wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

In some embodiments of compounds of structural Formula (I):
X, Y, and Z are each N;
R¹ and R² are each hydrogen;
R³ and R⁴ are each hydrogen;
R^{5a} and R^{5b} are each methyl, optionally substituted with one or more halo;
R^{5c} is —(CH₂)-phenyl, wherein R^{5c} is optionally substituted with one, two, three, or four substituents Q;
R^{5d} and R^{5e} are each hydrogen;
R⁶ is CHF₂; and
m is 0;
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
   wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

Provided herein is a compound of Formula (II):

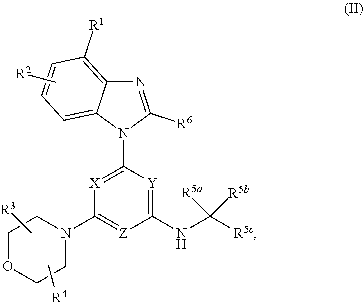

or an enantiomer, a mixture of enantiomers, a mixture of two or more diastereomers, or an isotopic variant thereof; or a pharmaceutically acceptable salt, solvate, hydrate, or prodrug thereof. In some embodiments, R^{5c} is C₆₋₁₄ aryl, optionally substituted with one or more substituents Q. In some embodiments, R^{5c} is phenyl, optionally substituted with one or more substituents Q. In some embodiments, R^{5c} is naphthyl, optionally substituted with one or more substituents Q. In some embodiments, R^{5c} is —(CR^{5f}R^{5g})ₙ—(C₆₋₁₄ aryl), wherein the aryl is optionally substituted with one or more substituents Q. In some embodiments, R_{5c} is —(CH₂)-phenyl, wherein the phenyl is optionally substituted with one or more substituents Q. In some embodiments, R^{5c} is —(CH₂)-naphthyl, wherein the naphthyl is optionally substituted with one or more substituents Q. In some embodiments, R^{5c} is heteroaryl, optionally substituted with one or more substituents Q. In some embodiments, R^{5c} is monocyclic heteroaryl, optionally substituted with one or more substituents Q. In some embodiments, R^{5c} is 5- or 6-membered heteroaryl, optionally substituted with one or more substituents Q. In some embodiments, R^{5c} is bicyclic heteroaryl, optionally substituted with one or more substituents Q. In some embodiments, R^{5c} is —(CR^{5f}R^{5g})ₙ-heteroaryl, wherein the heteroaryl is optionally substituted with one or more substituents Q. In some embodiments, R^{5c} is —(CR^{5f}R^{5g})ₙ-(monocyclic heteroaryl), wherein the heteroaryl is optionally substituted with one or more substituents Q, R^{5c} is —(CR^{5f}R^{5g})ₙ-(5- or 6-membered heteroaryl), wherein the heteroaryl is optionally substituted with one or more substituents Q. In some embodiments, R^{5c} is —(CR^{5f}R^{5g})ₙ-(bicyclic heteroaryl), wherein the heteroaryl is optionally substituted with one or more substituents Q.

Also provided herein is a compound of Formula (VII):

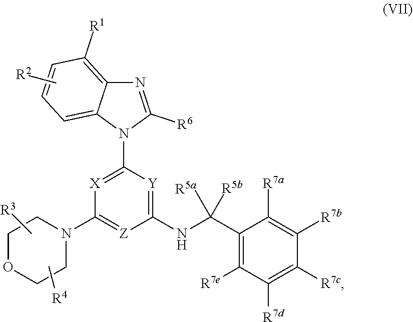

or an enantiomer, a mixture of enantiomers, a mixture of two or more diastereomers, or an isotopic variant thereof; or a pharmaceutically acceptable salt, solvate, hydrate, or prodrug thereof,
wherein:
R^{7a}, F^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one or more substituents Q.

Also provided herein is a compound of Formula (IX):

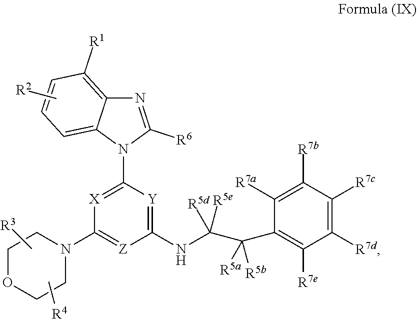

or an enantiomer, a mixture of enantiomers, a mixture of two or more diastereomers, or an isotopic variant thereof; or a pharmaceutically acceptable salt, solvate, hydrate, or prodrug thereof,
wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

In some embodiments, R^{7a} is hydrogen, halo, C₁₋₆ alkyl optionally substituted with one or more substituents Q, or —OR^{1a}.

In some embodiments, R^{7a} is hydrogen. In some embodiments, R^{7a} is (a) cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more substituents Q; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}. In some embodiments, R^{7a} is (i) halo; (ii) C₁₋₆ alkyl, C₆₋₁₄ aryl, C₇₋₁₅ aralkyl, heteroaryl, or heterocyclyl, each of which is optionally substituted with one or more substituents Q; or (iii) —OR^{1a} or —NR^{1b}R^{1c}.

In some embodiments, R^{7b} is hydrogen, halo, C₁₋₆ alkyl optionally substituted with one or more substituents Q, or —OR^{1a}. In some embodiments, R^{7b} is hydrogen.

In some embodiments, R^{7c} is hydrogen, halo, C₁₋₆ alkyl optionally substituted with one or more substituents Q, or —OR^{1a}. In some embodiments, R^{7c} is hydrogen, halo, or —OR^{1a}. In some embodiments, R^{7C} is chloro. In some embodiments, R^{7c} is —O—C₁₋₆ alkyl, optionally substituted with one or more substituents Q.

In some embodiments, R^{7d} is hydrogen, halo, C₁₋₆ alkyl optionally substituted with one or more substituents Q, or —OR^{1a}. In some embodiments, R^{7d} is hydrogen.

In some embodiments, R^{7e} is hydrogen, halo, C₁₋₆ alkyl optionally substituted with one or more substituents Q, or —OR^{1a}. In some embodiments, R^{7e} is hydrogen. In some embodiments, two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one or more substituents Q. In some embodiments, R^{7a} and R^{7b} together with the carbon atoms to which they are attached form C₆₋₁₄ aryl, optionally substituted with one or more substituents Q.

In some embodiments, R^{5a} is hydrogen. In some embodiments, R^{5a} is C₁₋₆ alkyl, optionally substituted with one or more substituents Q. In some embodiments, R^{5a} is hydrogen, methyl, or ethyl.

In some embodiments, R^{5b} is C₁₋₆ alkyl, optionally substituted with one or more substituents Q. In some embodiments, R^{5b} is methyl, ethyl, or propyl. In some embodiments, R^{5b} is —C(O)OR^{1a}. In some embodiments, R^{5b} is —C(O)O—C₁₋₆ alkyl. In some embodiments, R^{5b} is —C(O)OCH₃.

Also provided herein is a compound of Formula (X):

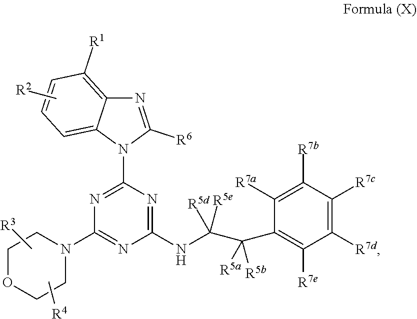

or an enantiomer, a mixture of enantiomers, a mixture of two or more diastereomers, or an isotopic variant thereof; or a pharmaceutically acceptable salt, solvate, hydrate, or prodrug thereof.

Provided herein is a compound of Formula (XI):

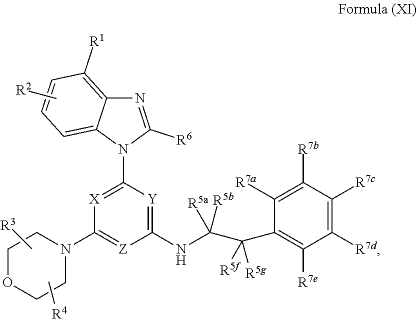

or an enantiomer, a mixture of enantiomers, a mixture of two or more diastereomers, or an isotopic variant thereof; or a pharmaceutically acceptable salt, solvate, hydrate, or prodrug thereof,
wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

In certain embodiments, R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}. In certain embodiments, one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; in certain embodiments, one of R^{7a}, R^{7b}, R^{7c}, R^{7d}, and R^{7e} is C₆₋₁₄ aryl, e.g., phenyl, optionally substituted with one, two, three, or four substituents Q^{a}; in certain embodiments, one of R^{7a}, R^{7b}, R^{7c}, R^{7d}, and R^{7e} is heteroaryl, e.g., 5-membered or 6-membered heteroaryl, optionally substituted with one, two, three, or four substituents Q^{a}; in certain embodiments, one of R^{7a}, R^{7b}, R^{7c}, R^{7d}, and R^{7e} is heterocyclyl, e.g., 5-membered or 6-membered heterocyclyl, optionally substituted with one, two, three, or four substituents Q^{a}; in certain embodiments, one of R^{7a}, R^{7b}, R^{7c}, R^{7d}, and R^{7e} is phenyl, imidazolyl, pyrozolyl, pyridinyl, piperidinyl, or piperazinyl, each optionally substituted with one, two, three, or four substituents Q^{a}; in certain embodiments, one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one or more substituents Q^{a}; in certain embodiments, one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and in certain embodiments, one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, l-ethylpiperidin-4-yl, 1-isopropylpiperidin-4-yl, l-acetylpiperidin-4-yl, l-methylsulfonylpiperidin-4-yl, or 4-methylpiperazin-1-yl.

In certain embodiments, R^{7a} is C₆₋₁₄ aryl, heteroaryl, or heterocyclyl, each of which is optionally substituted with one, two, three, or four substituents Q^{a}; in certain embodiments, R^{7a} is C₆₋₁₄ aryl, e.g., phenyl, optionally substituted with one, two, three, or four substituents Q^{a}; in certain embodiments, R^{7a} is heteroaryl, e.g., 5-membered or 6-membered heteroaryl, optionally substituted with one, two, three, or four substituents Q^{a}; in certain embodiments, R^{7a} is heterocyclyl, e.g., 5-membered or 6-membered heterocyclyl, optionally substituted with one, two, three, or four substituents Q^{a}; in certain embodiments, R^{7a} is phenyl, imidazolyl, pyrozolyl, pyridinyl, piperidinyl, or piperazinyl, each optionally substituted with one, two, three, or four substituents Q^{a}; in certain embodiments, R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, l-methylpyrrolidin-3-yl, piperidin-4-yl, 1-methylpiperidin-4-yl, l-ethylpiperidin-4-yl, l-isopropylpiperidin-4-yl, 1-acetylpiperidin-4-yl, 1-methylsulfonylpiperidin-4-yl, or 4-methylpiperazin-1-yl.

In certain embodiments:

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently C₁₋₆ alkyl, optionally substituted with one, two, three, four, or five substituents Q;

R^{5f} and R^{5g} are each independently hydrogen, halo, C₁₋₆ alkyl, optionally substituted with one, two, three, four, or five substituents Q; or R^{5f} and R^{5g} together with the carbon atom to which they are attached form C₁₋₁₀ cycloalkyl or heterocyclyl, each of which is optionally substituted with one, two, three, four, or five substituents Q;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CR^{x}, with the proviso that at least two of X, Y, and Z are N; where R^{x} is a hydrogen or C₁₋₆ alkyl, optionally substituted with one, two, three, or four substituents Q^{a}.

In certain embodiments:

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently C₁₋₆ alkyl;

R^{5f} and R^{5g} are each independently hydrogen or C₁₋₆ alkyl; or R^{5f} and R^{5g} together with the carbon atom to which they are attached form C₁₋₁₀ cycloalkyl;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments:

R¹ is hydrogen or methoxy;

R² is hydrogen;

R³ and R⁴ are hydrogen;

R⁶ is difluoromethyl;

R^{5a} and R^{5b} are methyl;

R^{5f} and R^{5g} are hydrogen; or R^{5f} and R^{5g} together with the carbon atom to which they are attached form cyclopropyl, cyclobutyl, cyclopentyl, or cyclohexyl;
R^{7a} is C₆₋₁₄ aryl, monocyclic heteroaryl, or monocyclic heterocyclyl, each of which is optionally substituted with one, two, three, or four substituents Q^{a};
R^{7b}, R^{7c}, R^{7d}, and R^{7e} are hydrogen; and X, Y, and Z are each independently N or CH.

In certain embodiments:

R¹ is hydrogen or methoxy;

R² is hydrogen;

R³ and R⁴ are hydrogen;

R⁶ is difluoromethyl;

R^{5a} and R^{5b} are methyl;

R^{5f} and R^{5g} are hydrogen; or R^{5f} and R^{5g} together with the carbon atom to which they are attached form cyclopropyl, cyclobutyl, cyclopentyl, or cyclohexyl;

R^{7a} is phenyl, 5- or 6-membered heteroaryl, or 5- or 6-membered heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments:

R¹ is hydrogen or methoxy;

R² is hydrogen;

R³ and R⁴ are hydrogen;

R⁶ is difluoromethyl;

R^{5a} and R^{5b} are methyl;

R^{5f} and R^{5g} are hydrogen; or R^{5f} and R^{5g} together with the carbon atom to which they are attached form cyclopropyl, cyclobutyl, cyclopentyl, or cyclohexyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments, R^{7a} is phenyl, imidazolyl, pyrozolyl, pyridinyl, piperidinyl, or piperazinyl, each of which is optionally substituted with one, two, three, or four substituents Q^{a}.

Provided herein is a compound of Formula (XVI):

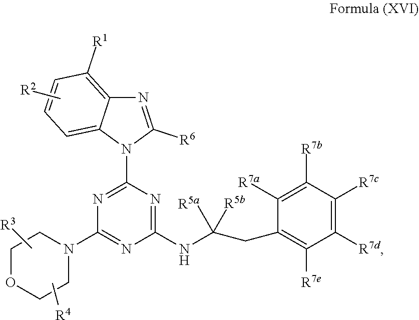

or an enantiomer, a mixture of enantiomers, a mixture of two or more diastereomers, or an isotopic variant thereof; or a pharmaceutically acceptable salt, solvate, hydrate, or prodrug thereof.

In some embodiments, R^{5a} is C₁₋₆ alkyl, optionally substituted with one or more substituents Q. In some embodiments, R^{5a} is methyl.

In some embodiments, R^{5b} is C₁₋₆ alkyl, optionally substituted with one or more substituents Q. In some embodiments, R^{5b} is methyl.

In some embodiments, R^{5a} and R^{5b} are methyl.

In some embodiments, R^{7a} is hydrogen, halo, C₁₋₆ alkyl, C₆₋₄ aryl, heteroaryl, or heterocyclyl, where the alkyl, aryl, heteroaryl, and heterocyclyl are each optionally substituted with one or more substituents Q. In some embodiments, R^{7a} is C₆₋₄ aryl, optionally substituted with one or more substituents Q. In some embodiments, R^{7a} is phenyl, optionally substituted with one or more substituents Q In some embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-florophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, or 3-morpholin-4-ylmethylphenyl. In some embodiments, R^{7a} is heteroaryl, optionally substituted with one or more substituents Q. In some embodiments, R^{7a} is monocyclic heteroaryl, optionally substituted with one or more substituents Q. In some embodiments, R^{7a} is 5- or 6-membered heteroaryl, each optionally substituted with one or more substituents Q. In some embodiments, R^{7a} is imidazolyl, pyrozolyl, pyridinyl, or pyrimidinyl, each optionally substituted with one or more substituents Q. In some embodiments, R^{7a} is imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl. In some embodiments, R^{7a} is heterocyclyl, optionally substituted with one or more substituents Q. In some embodiments, R^{7a} is monocyclic heterocyclyl, optionally substituted with one or more substituents Q. In some embodiments, R^{7a} is 5- or 6-membered heterocyclyl, each optionally substituted with one or more substituents Q. In some embodiments, R^{7a} is pyrrolidinyl, piperidinyl, or piperazinyl, each optionally substituted with one or more substituents Q. In some embodiments, R^{7a} is pyrrolidin-3-yl, l-methylpyrrolidin-3-yl, piperidin-4-yl, l-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In some embodiments, R^{7b} is hydrogen, halo, or C₁₋₆ alkyl optionally substituted with one or more substituents Q. In some embodiments, R^{7b} is hydrogen.

In some embodiments, R^{7c} is hydrogen, halo, or C₁₋₆ alkyl optionally substituted with one or more substituents Q. In some embodiments, R^{7c} is hydrogen.

In some embodiments, R^{7d} is hydrogen, halo, or C₁₋₆ alkyl optionally substituted with one or more substituents Q. In some embodiments, R^{7d} is hydrogen.

In some embodiments, R^{7e} is hydrogen, halo, or C₁₋₆ alkyl optionally substituted with one or more substituents Q. In some embodiments, R^{7e} is hydrogen.

In some embodiments, R^{7a} is C₆₋₄ aryl, heteroaryl, or heterocyclyl, each optionally substituted with one or more substituents Q; and R^{7b}, R^{7c}, R^{7d}, and R^{7e} are hydrogen.

In one embodiment of a compound of Formula (XVI), one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In another embodiment of a compound Formula (XVI), one of R^{7a}, R^{7b}, R^{7c}, R^{7d}, and R^{7e} is C₆₋₁₄ aryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formulae (XVI), one of R^{7a}, R^{7b}, R^{7c}, R^{7d}, and R^{7e} is heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is 5-membered or 6-membered heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, l-methylpyrrolidin-3-yl, piperidin-4-yl, 1-methylpiperidin-4-yl, l-ethylpiperidin-4-yl, l-isopropylpiperidin-4-yl, 1-acetylpiperidin-4-yl, 1-methylsulfonylpiperidin-4-yl, or 4-methylpiperazin-1-yl.

In still another embodiment of a compound of Formula (XVI), one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVI), R^{7a} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is 5-membered or 6-membered heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, l-ethylpiperidin-4-yl, 1-isopropylpiperidin-4-yl, l-acetylpiperidin-4-yl, l-methylsulfonylpiperidin-4-yl, or 4-methylpiperazin-1-yl.

In yet another embodiment of a compound of Formula (XVI), R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, l-methylpiperidin-4-yl, or 4-methylpiperazin-1-yl; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVI),

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently C₁₋₆ alkyl, optionally substituted with one, two, three, four, or five substituents Q;

R^{7a} is C₆₋₁₄ aryl, heteroaryl, or heterocyclyl, each of which is optionally substituted with one or more substituents Q^{a}; and

R^{7b}, R^{7c}, R^{7d}, and R^{7e} are hydrogen.

In one embodiment of a compound of Formula (XVI):

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently C₁₋₆ alkyl;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVI):

R¹ is hydrogen or methoxy;

R² is hydrogen;

R³ and R⁴ are hydrogen;

R⁶ is difluoromethyl;

R^{5a} and R^{5b} are methyl;

R^{7a} is phenyl, imidazolyl, pyrozolyl, pyridinyl, pyrimidinyl, pyrrolidinyl, piperidinyl, or piperazinyl, each of which is optionally substituted with one, two, three, four, or five substituents Q; and

R^{7b}, R^{7c}, R^{7d}, and R^{7e} are hydrogen.

In one embodiment of a compound of Formula (XVI), R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; and R¹, R², R³, R⁴, R⁶, R^{7a}, R^{7b}, R^{7c}, R^{7d}, R^{7e}, R^{1a}, R^{1b}, R^{1c}, and R^{1d} are defined herein elsewhere.

In one embodiment of any of the formulae provided herein:

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently hydrogen or C₁₋₆ alkyl optionally substituted with one, two, three, four, or five substituents Q;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CR^{x}, with the proviso that at least two of X, Y, and Z are N; where R^{x} is a hydrogen or C₁₋₆ alkyl, optionally substituted with one, two, three, or four substituents Q^{a}.

In one embodiment of any of the formulae provided herein:

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently hydrogen or C₁₋₆ alkyl;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein:

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is C₆₋₁₄ aryl, monocyclic heteroaryl, or monocyclic heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein:

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, 5- or 6-membered heteroaryl, or 5- or 6-membered heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein:

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein:

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, piperidinyl, or piperazinyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein, R¹ is hydrogen. In one embodiment of any of the formulae provided herein, R¹ is —OR^{1a}. In one embodiment of any of the formulae provided herein, R¹ is —O—C₁₋₆ alkyl. In one embodiment of any of the formulae provided herein, R¹ is methoxy.

In one embodiment of any of the formulae provided herein, R² is hydrogen. In one embodiment of any of the formulae provided herein, R² is —NR^{1b}R^{1c}. In one embodiment of any of the formulae provided herein, R² is amino.

In one embodiment of any of the formulae provided herein, R³ is hydrogen.

In one embodiment of any of the formulae provided herein, R⁴ is hydrogen.

In one embodiment of any of the formulae provided herein, R⁶ is C₁₋₆ alkyl, optionally substituted with one or more substituents Q.

In one embodiment of any of the formulae provided herein, R⁶ is methyl, fluoromethyl, difluoromethyl, or trifluoromethyl. In one embodiment of any of the formulae provided herein, R⁶ is difluoromethyl.

The groups or variables, R¹, R², R³, R⁴, R⁶, R^{5a}, R^{5b}, R^{5c}, R^{5d}, R^{5e}, R^{5f}, R^{5g}, R^{7a}, R^{7b}, R^{7c}, R^{7d}, R^{7e}, m, n, X, Y, and Z in Formulae provided herein, e.g., Formulae (I), (II), (VII), (IX), (X), (XI), (XVI), are further defined in the embodiments described herein. All combinations of the embodiments provided herein for such groups and/or variables are within the scope of this disclosure.

In certain embodiments, m is 0. In certain embodiments, m is 1.

In certain embodiments, n is 0. In certain embodiments, n is 1. In certain embodiments, n is 2. In certain embodiments, n is 3. In certain embodiments, n is 4. In certain embodiments, n is 0, 1, or 2. In certain embodiments, n is 0, 1, 2, or 3. In certain embodiments, n is 1, 2, or 3. In certain embodiments, n is 1 or 2.

In certain embodiments, m is 0, and n is 0, 1, 2, or 3. In certain embodiments, m is 0, n is 0, 1, or 2. In certain embodiments, m is 0, n is 0 or 1. In certain embodiments, m is 0, n is 0. In certain embodiments, m is 0 and n is 1. In certain embodiments, m is 1, n is 0, 1, 2, or 3. In certain embodiments, m is 1, n is 0, 1, or 2. In certain embodiments, m is 1, n is 0 or 1. In certain embodiments, m is 1, n is 0. In certain embodiments, m is 1, n is 1.

In specific embodiments, m is 0, n is 1, and R^{5a} and R^{5b} are each methyl.

In certain embodiments, X is N. In certain embodiments, X is CR^{x}, wherein R^{x} is as defined herein. In certain embodiments, X is CH.

In certain embodiments, Y is N. In certain embodiments, Y is CR^{x}, wherein R^{x} is as defined herein. In certain embodiments, Y is CH.

In certain embodiments, Z is N. In certain embodiments, Z is CR^{x}, wherein R^{x} is as defined herein. In certain embodiments, Z is CH.

In certain embodiments, X, Y, and Z are N. In certain embodiments, X and Y are N, and Z is CH.

In certain embodiments, X and Z are N, and Y is CH. In certain embodiments, Y and Z are N, and X is CH.

In certain embodiments, the compound provided herein is not 4-(2-(difluoromethyl)-1H-benzo[d]imidazol-1-yl)-6-morpholino-N-(2-phenyl-2-(pyrrolidin-1-yl)ethyl)-1,3,5-triazin-2-amine. In certain embodiments, the compound provided herein is not 6-(2-(difluoromethyl)-1H-benzo[d]imidazol-1-yl)-N-(1-(4-((R)-3-(methoxymethyl)morpholino)phenyl)ethyl)-2-morpholinopyrimidin-4-amine.

In certain embodiments, when X, Y, and Z are N, and R^{5a} is hydrogen, R^{5b} is not heterocyclyl. In certain embodiments, when X, Y, and Z are N, and R^{5a} is hydrogen, R^{5b} is not 5-membered heterocyclyl. In certain embodiments, when X, Y, and Z are N, and R^{5a} is hydrogen, R^{5b} is not pyrrolidinyl. In certain embodiments, when X, Y, and Z are N, and R^{5a} is hydrogen, R^{5b} is not pyrrolidin-1-yl.

In certain embodiments, when X and Z are N, Y is CH, and R^{5a} is hydrogen, R^{5b} is morpholino-substituted phenyl. In certain embodiments, when X and Z are N, Y is CH, and R^{5a} is hydrogen, R^{5b} is not 4-((R)-3-(methoxymethyl)morpholino)phenyl.

In one embodiment, provided herein is a compound selected from:

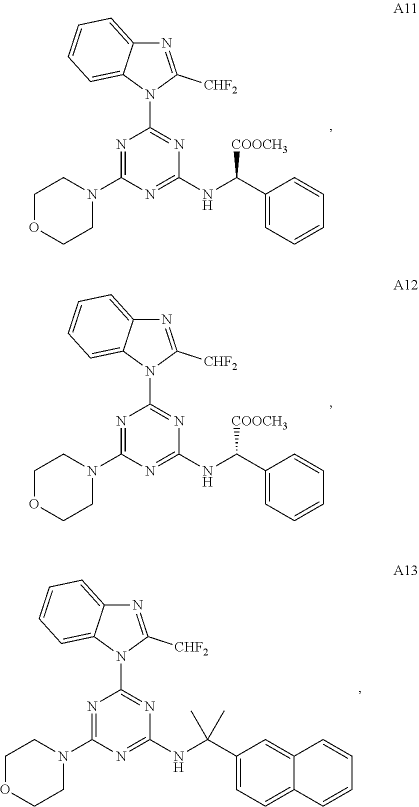 [SMILES: COC(=O)[C@H](Nc1nc(N2CCOCC2)nc(-n2c(C(F)F)nc3ccccc32)n1)c1ccccc1 ; "," ; COC(=O)[C@@H](Nc1nc(N2CCOCC2)nc(-n2c(C(F)F)nc3ccccc32)n1)c1ccccc1 ; "," ; CC(C)(Nc1nc(N2CCOCC2)nc(-n2c(C(F)F)nc3ccccc32)n1)c1ccc2ccccc2c1 ; ","]

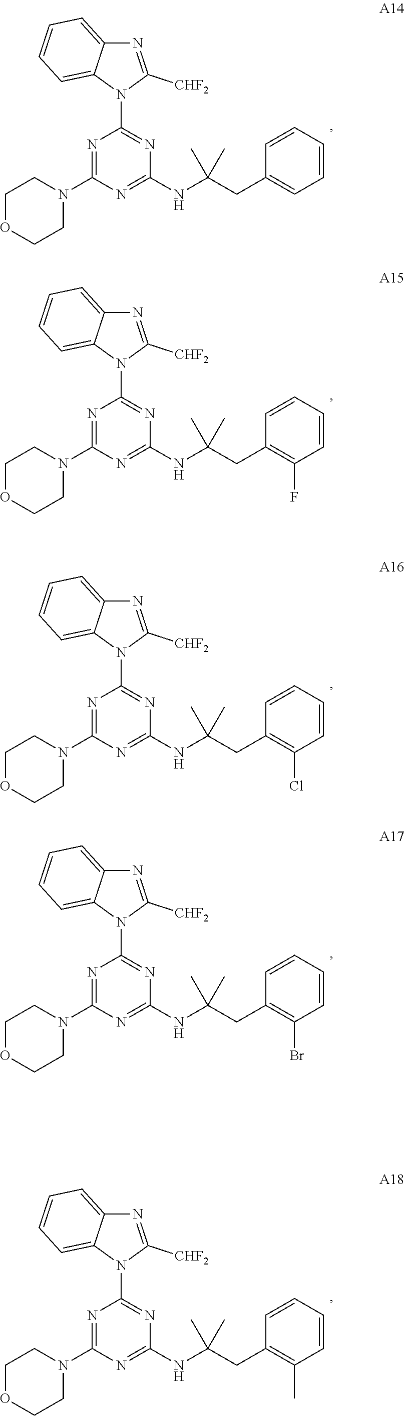

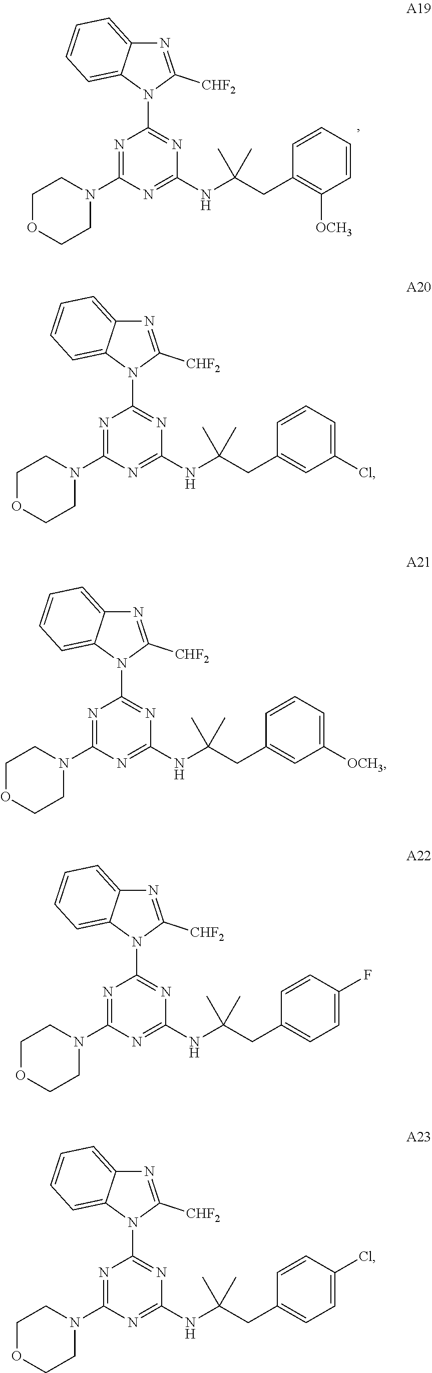 [SMILES: COc1ccccc1CC(C)(C)Nc1nc(N2CCOCC2)nc(-n2c(C(F)F)nc3ccccc32)n1 ; "," ; CC(C)(Cc1cccc(Cl)c1)Nc1nc(N2CCOCC2)nc(-n2c(C(F)F)nc3ccccc32)n1 ; COc1cccc(CC(C)(C)Nc2nc(N3CCOCC3)nc(-n3c(C(F)F)nc4ccccc43)n2)c1 ; CC(C)(Cc1ccc(F)cc1)Nc1nc(N2CCOCC2)nc(-n2c(C(F)F)nc3ccccc32)n1 ; CC(C)(Cc1ccc(Cl)cc1)Nc1nc(N2CCOCC2)nc(-n2c(C(F)F)nc3ccccc32)n1]

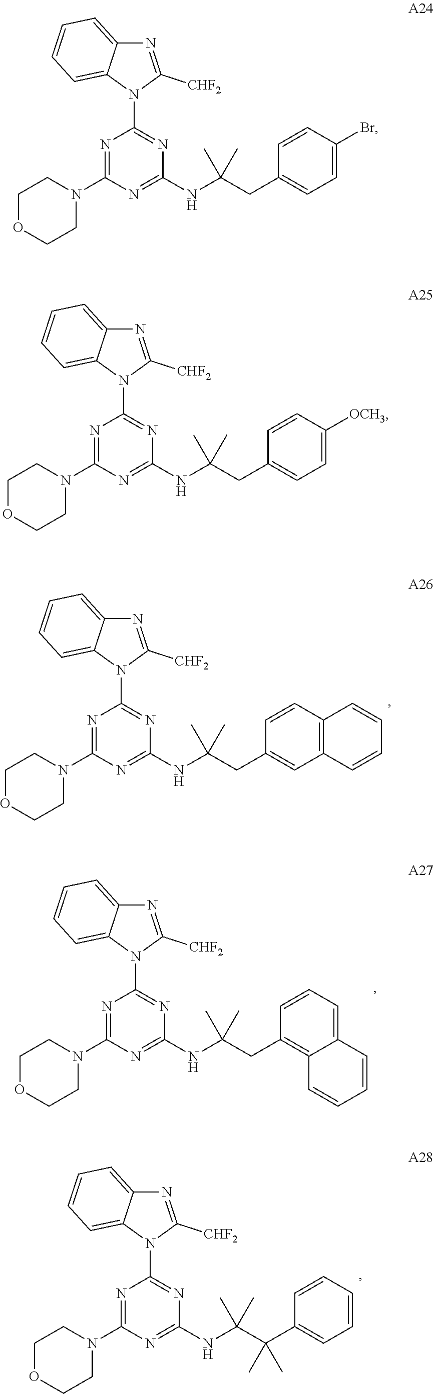

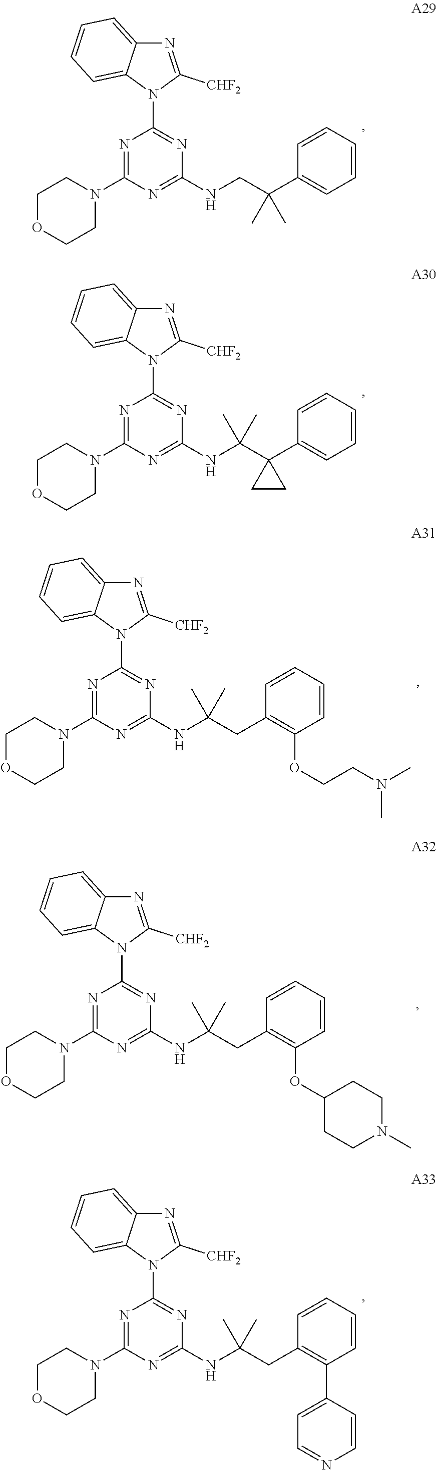

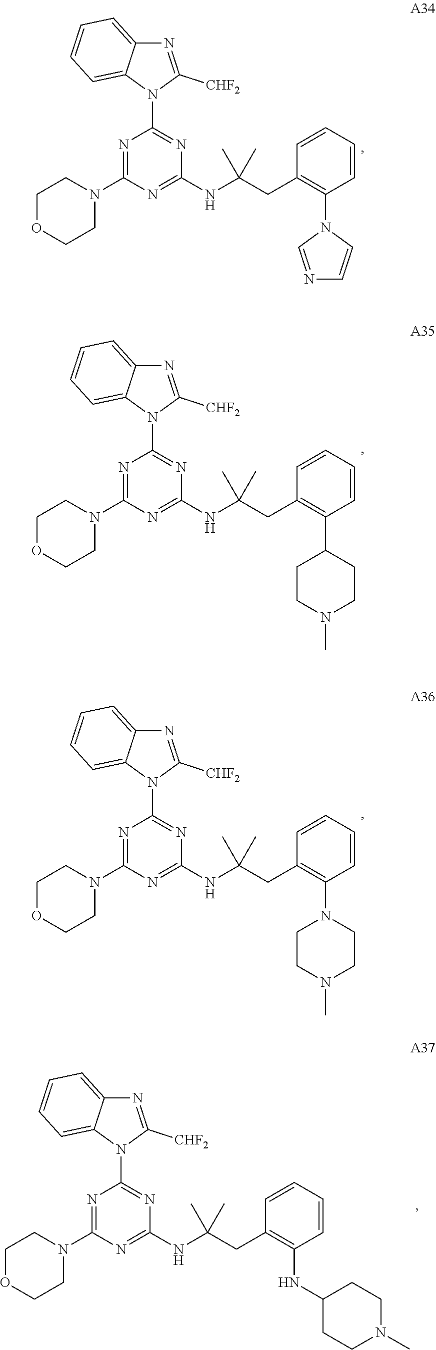

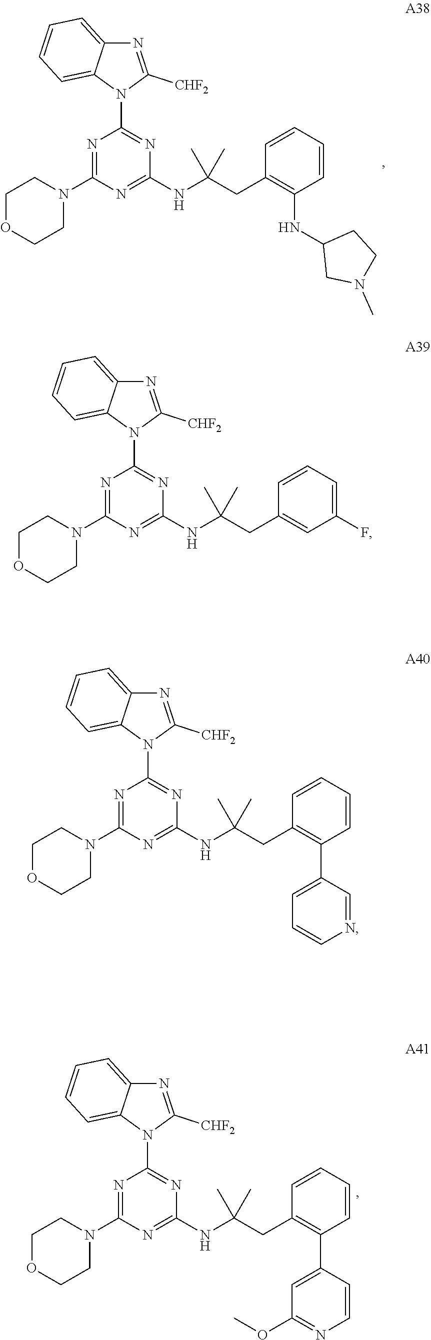 [SMILES: CN1CCC(Nc2ccccc2CC(C)(C)Nc2nc(N3CCOCC3)nc(-n3c(C(F)F)nc4ccccc43)n2)C1 ; "," ; CC(C)(Cc1cccc(F)c1)Nc1nc(N2CCOCC2)nc(-n2c(C(F)F)nc3ccccc32)n1 ; CC(C)(Cc1ccccc1-c1cccnc1)Nc1nc(N2CCOCC2)nc(-n2c(C(F)F)nc3ccccc32)n1 ; COc1cc(-c2ccccc2CC(C)(C)Nc2nc(N3CCOCC3)nc(-n3c(C(F)F)nc4ccccc43)n2)ccn1 ; ","]

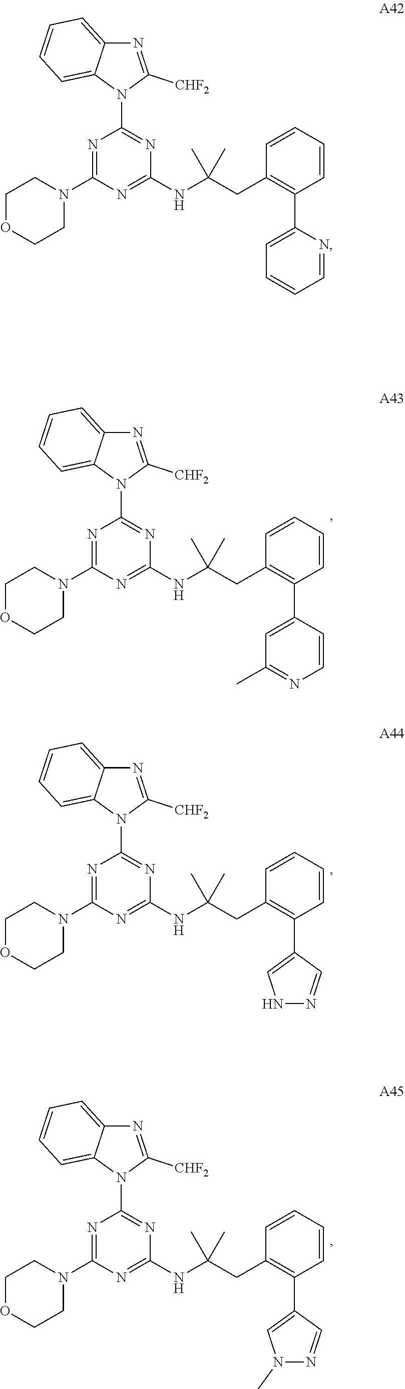

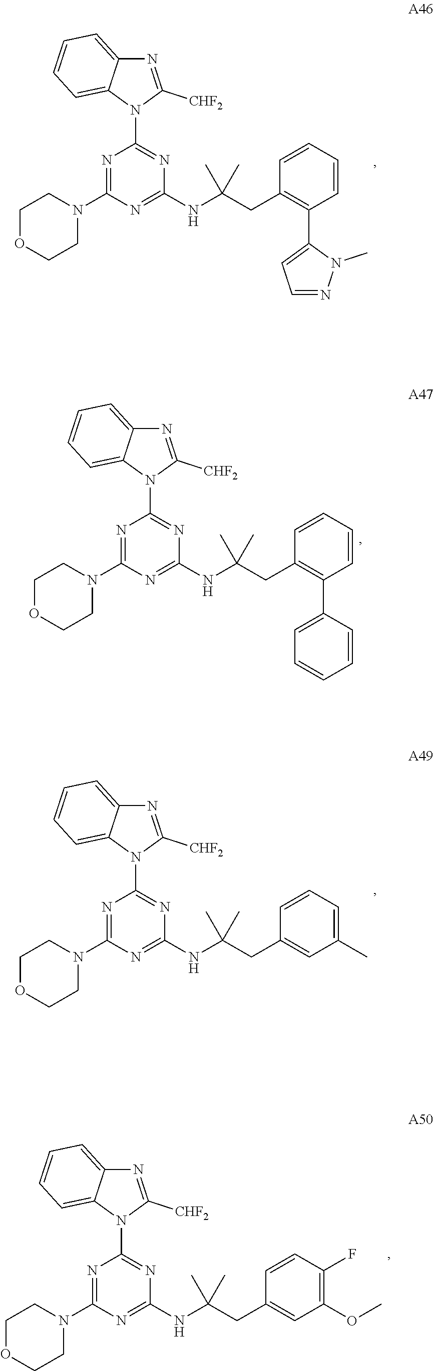

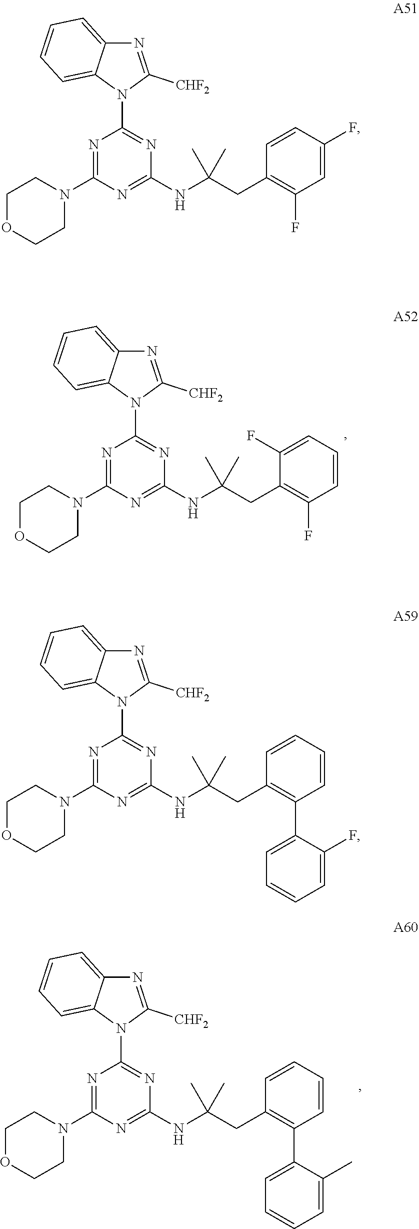 [SMILES: CC(C)(Cc1ccc(F)cc1F)Nc1nc(N2CCOCC2)nc(-n2c(C(F)F)nc3ccccc32)n1 ; CC(C)(Cc1c(F)cccc1F)Nc1nc(N2CCOCC2)nc(-n2c(C(F)F)nc3ccccc32)n1 ; "," ; CC(C)(Cc1ccccc1-c1ccccc1F)Nc1nc(N2CCOCC2)nc(-n2c(C(F)F)nc3ccccc32)n1 ; Cc1ccccc1-c1ccccc1CC(C)(C)Nc1nc(N2CCOCC2)nc(-n2c(C(F)F)nc3ccccc32)n1 ; ","]

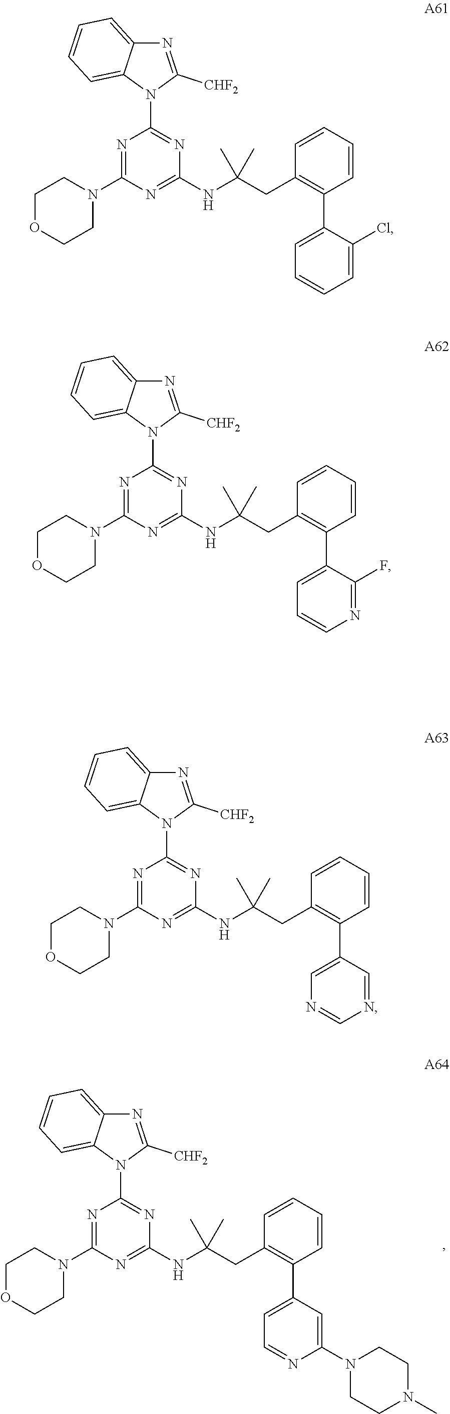 [SMILES: CC(C)(Cc1ccccc1-c1ccccc1Cl)Nc1nc(N2CCOCC2)nc(-n2c(C(F)F)nc3ccccc32)n1 ; CC(C)(Cc1ccccc1-c1cccnc1F)Nc1nc(N2CCOCC2)nc(-n2c(C(F)F)nc3ccccc32)n1 ; CC(C)(Cc1ccccc1-c1cncnc1)Nc1nc(N2CCOCC2)nc(-n2c(C(F)F)nc3ccccc32)n1 ; CN1CCN(c2cc(-c3ccccc3CC(C)(C)Nc3nc(N4CCOCC4)nc(-n4c(C(F)F)nc5ccccc54)n3)ccn2)CC1 ; ","]

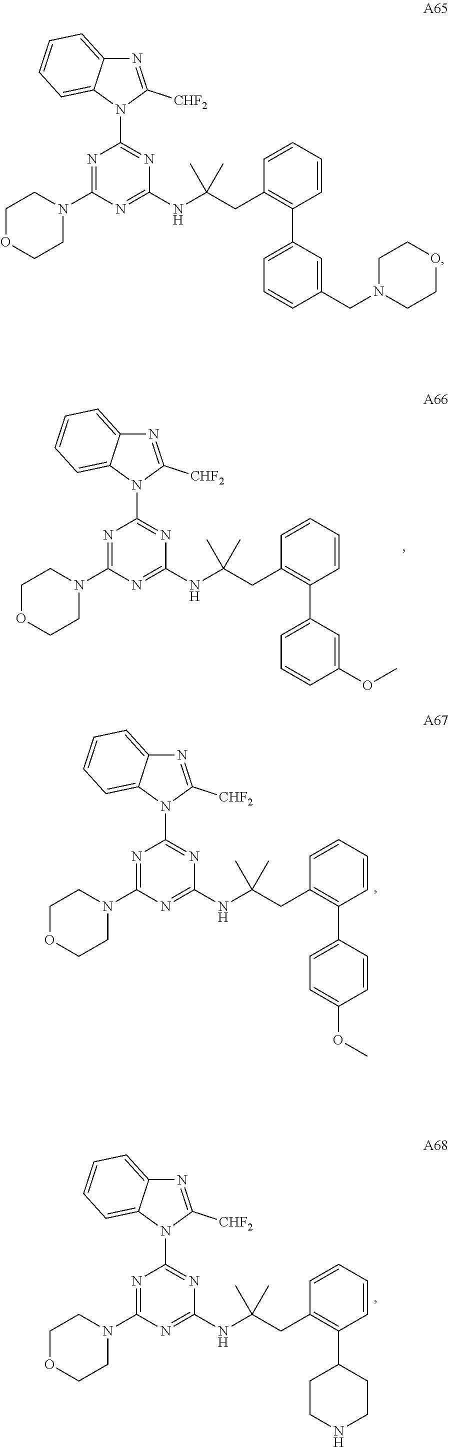

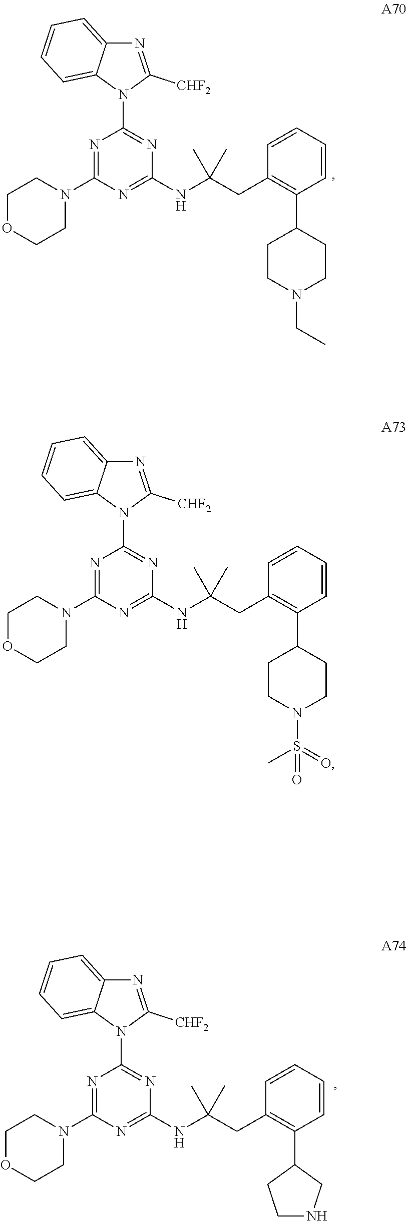 [SMILES: CCN1CCC(c2ccccc2CC(C)(C)Nc2nc(N3CCOCC3)nc(-n3c(C(F)F)nc4ccccc43)n2)CC1 ; "," ; CC(C)(Cc1ccccc1C1CCN(S(C)(=O)=O)CC1)Nc1nc(N2CCOCC2)nc(-n2c(C(F)F)nc3ccccc32)n1 ; CC(C)(Cc1ccccc1C1CCNC1)Nc1nc(N2CCOCC2)nc(-n2c(C(F)F)nc3ccccc32)n1 ; ","]

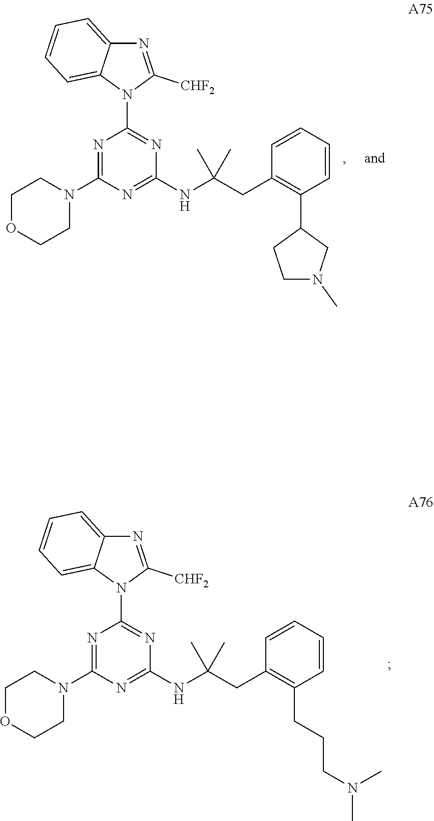 [SMILES: CN1CCC(c2ccccc2CC(C)(C)Nc2nc(N3CCOCC3)nc(-n3c(C(F)F)nc4ccccc43)n2)C1 ; "," ; "and" ; CN(C)CCCc1ccccc1CC(C)(C)Nc1nc(N2CCOCC2)nc(-n2c(C(F)F)nc3ccccc32)n1 ; ";"]

or an isotopic variant, pharmaceutically acceptable salt, solvate, hydrate, or prodrug thereof.

In one embodiment, the PI3K inhibitor is Compound A35, or an isotopic variant, pharmaceutically acceptable salt, solvate, hydrate, or prodrug thereof. In one embodiment, the PI3K inhibitor is Compound A36, or an isotopic variant, pharmaceutically acceptable salt, solvate, hydrate, or prodrug thereof. In one embodiment, the PI3K inhibitor is Compound A68, or an isotopic variant, pharmaceutically acceptable salt, solvate, hydrate, or prodrug thereof. In one embodiment, the PI3K inhibitor is Compound A70, or an isotopic variant, pharmaceutically acceptable salt, solvate, hydrate, or prodrug thereof. In one embodiment, the PI3K inhibitor is Compound A37, or an isotopic variant, pharmaceutically acceptable salt, solvate, hydrate, or prodrug thereof. In one embodiment, the PI3K inhibitor is Compound A38, or an isotopic variant, pharmaceutically acceptable salt, solvate, hydrate, or prodrug thereof. In one embodiment, the PI3K inhibitor is Compound A41, or an isotopic variant, pharmaceutically acceptable salt, solvate, hydrate, or prodrug thereof. In one embodiment, the PI3K inhibitor is Compound A42, or an isotopic variant, pharmaceutically acceptable salt, solvate, hydrate, or prodrug thereof. In one embodiment, the PI3K inhibitor is Compound A43, or an isotopic variant, pharmaceutically acceptable salt, solvate, hydrate, or prodrug thereof. In one embodiment, the PI3K inhibitor is Compound A44, or an isotopic variant, pharmaceutically acceptable salt, solvate, hydrate, or prodrug thereof. In one embodiment, the PI3K inhibitor is Compound A62, or an isotopic variant, pharmaceutically acceptable salt, solvate, hydrate, or prodrug thereof. In one embodiment, the PI3K inhibitor is Compound A63, or an isotopic variant, pharmaceutically acceptable salt, solvate, hydrate, or prodrug thereof. In one embodiment, the PI3K inhibitor is Compound A64, or an isotopic variant, pharmaceutically acceptable salt, solvate, hydrate, or prodrug thereof. In one embodiment, the PI3K inhibitor is Compound A65, or an isotopic variant, pharmaceutically acceptable salt, solvate, hydrate, or prodrug thereof. In one embodiment, the PI3K inhibitor is Compound A66, or an isotopic variant, pharmaceutically acceptable salt, solvate, hydrate, or prodrug thereof. In one embodiment, the PI3K inhibitor is Compound A67, or an isotopic variant, pharmaceutically acceptable salt, solvate, hydrate, or prodrug thereof.

Synthesis of compounds of any of the Formulae provided herein e.g., Formulae (I), (II), (VII), (IX), (X), (XI), (XVI), is described in U.S. Pat. No. 9,056,852 B2, which is incorporated by reference for such disclosure.

### Uses and Methods of Treatment

Some embodiments provided herein describe a method for treating a patient with B cell maligancies, the method comprising administering to the patient in need thereof an effective amount of a PI3K inhibitor having the structure of Formula (I). Also provided herein, in some embodiments, is a method of preventing relapse in a patient with B cell maligancies, the method comprising administering the patient in need thereof with an effective amount of a PI3K inhibitor having the structure of of Formula (I). In some embodiments provided herein is a method for achieving and retaining partial cancer remission in a patient with B cell maligancies, the method comprising administering to the patient in need thereof an effective amount of a PI3K inhibitor having the structure of Formula (I). In some embodiments provided herein is a method for achieving and retaining complete cancer remission in a patient with B cell maligancies, the method comprising administering to the patient in need thereof an effective amount of a PI3K inhibitor of Formula (I).

In some embodiments, the methods, including dosing regimens and schedules, described herein avoid or reduce adverse or unwanted side effects associated with the use of a PI3K inhibitor. In some embodiments, the methods described herein avoid, reduce, or minimize the risk of death due to infections. In some embodiments, the methods described herein avoid, reduce, or minimize infections, neutropenia, diarrhea/colitis, elevated liver transaminases (alanine aminotransferase/aspartate aminotransferase >5× upper limit of normal), pneumonitis, rash, hepatic impairment, renal impairment, pyrexia, or increased triglycerides, or a combination thereof in patients receiving the treatment described herein. In certain embodiments, the methods described herein avoid, reduce, or minimize the incidence of infection. In certain embodiments, the methods described herein avoid, reduce, or minimize the incidence of neutropenia. In certain embodiments, the methods described herein avoid, reduce, or minimize the incidence of diarrhea/colitis. In certain embodiments, the methods described herein avoid, reduce, or minimize the incidence of elevated liver transaminases. In certain embodiments, the methods described herein avoid, reduce, or minimize the incidence of pneumonitis. In certain embodiments, the methods described herein avoid, reduce, or minimize the incidence of a rash. In certain embodiments, the methods described herein avoid, reduce, or minimize the incidence of hepatic impairment or renal impairment. In certain embodiments, the methods described herein avoid, reduce, or minimize the incidence of pyrexia. In certain embodiments, the methods described herein avoid, reduce, or minimize the incidence of increased triglycerides. In certain embodiments, the methods described herein avoid, reduce, or minimize enterocolitis (manifested as diarrhea), cutaneous toxicities, liver toxicity (manifested as elevation of transaminases), pulmonary toxicity (manifested as non-infectious pneumonitis), infections, or combinations thereof.

In some embodiments, the methods described herein provides a high objective response rate (ORR) as determined by tumor assessment from radiological tests and/or physical examination. In some embodiments, the methods described herein provide a durable response (DR) and/or increased durable response rate (DRR; a continuous response [complete or partial objective response] beginning within 12 months of treatment and lasting ≥6 months) in the subject or patient. In some embodiments, the methods described herein provide complete remission. In some embodiments, the methods described herein provide complete remission beginning within 12 months of treatment and lasting ≥6 months. In some embodiments, the methods described herein provide a complete response (CR) and/or no evidence of disease (NED) beginning within 12 months of treatment and lasting ≥6 months.

In some embodiments of a method of treating B cell maligancies including relapsed or refractory B cell malignancies and relapsed or refractory follicular lymphoma (FL), the discontinuation rate due to adverse events is less than 25%, less than 20%, less than 15%, less than 10%, less than 8%, less than 5%.

The “discontinuation rate” is defined as the number of subjects who discontinue the study drugs prior to the study completion divided by the number of subjects treated.

In some embodiments, the discontinuation rate due to adverse events is less than 25%, less than 20%, less than 15%, less than 10%, less than 8%, less than 5%. In some embodiments, the discontinuation rate due to adverse events is less than 25%. In some embodiments, the discontinuation rate due to adverse events is less than 20%. In some embodiments, the discontinuation rate due to adverse events is less than 15%. In some embodiments, the discontinuation rate due to adverse events is less than 10%. In some embodiments, the discontinuation rate due to adverse events is less than 8%. In some embodiments, the discontinuation rate due to adverse events is about 4%.

In some embodiments, the discontinuation rate due to adverse events when the subjects are administered a compound of Formula (I), or an isotopic variant thereof or a pharmaceutically acceptable salt, solvate, hydrate, or prodrug is less for subjects on an intermittent dosing schedule (IS) than the discontinuation rate observed for subjects on a continuous dosing schedule (CS).

In certain embodiments, provided herein are methods for treating or preventing a disease comprising administering an effective amount of a compound of Formula (I), or an isotopic variant thereof or a pharmaceutically acceptable salt, solvate, hydrate, or prodrug. In some embodiments of the methods provided herein, the compound of Formula (I) is Compound A35 or an isotopic variant, pharmaceutically acceptable salt, solvate, hydrate, or prodrug thereof. In some embodiments of the methods provided herein, the compound of Formula (I) is Compound A36 or an isotopic variant, pharmaceutically acceptable salt, solvate, hydrate, or prodrug thereof. In some embodiments of the methods provided herein, the compound of Formula (I) is Compound A68 or an isotopic variant, pharmaceutically acceptable salt, solvate, hydrate, or prodrug thereof. In some embodiments of the methods provided herein, the compound of Formula (I) is Compound A70 or an isotopic variant, pharmaceutically acceptable salt, solvate, hydrate, or prodrug thereof. In some embodiments of the methods provided herein, the compound of Formula (I) is Compound A37 or an isotopic variant, pharmaceutically acceptable salt, solvate, hydrate, or prodrug thereof. In some embodiments of the methods provided herein, the compound of Formula (I) is Compound A38 or an isotopic variant, pharmaceutically acceptable salt, solvate, hydrate, or prodrug thereof. In some embodiments of the methods provided herein, the compound of Formula (I) is Compound A41 or an isotopic variant, pharmaceutically acceptable salt, solvate, hydrate, or prodrug thereof. In some embodiments of the methods provided herein, the compound of Formula (I) is Compound A42 or an isotopic variant, pharmaceutically acceptable salt, solvate, hydrate, or prodrug thereof. In some embodiments of the methods provided herein, the compound of Formula (I) is Compound A43 or an isotopic variant, pharmaceutically acceptable salt, solvate, hydrate, or prodrug thereof. In some embodiments of the methods provided herein, the compound of Formula (I) is Compound A44 or an isotopic variant, pharmaceutically acceptable salt, solvate, hydrate, or prodrug thereof. In some embodiments of the methods provided herein, the compound of Formula (I) is Compound A62 or an isotopic variant, pharmaceutically acceptable salt, solvate, hydrate, or prodrug thereof. In some embodiments of the methods provided herein, the compound of Formula (I) is Compound A63 or an isotopic variant, pharmaceutically acceptable salt, solvate, hydrate, or prodrug thereof. In some embodiments of the methods provided herein, the compound of Formula (I) is Compound A64 or an isotopic variant, pharmaceutically acceptable salt, solvate, hydrate, or prodrug thereof. In some embodiments of the methods provided herein, the compound of Formula (I) is Compound A65 or an isotopic variant, pharmaceutically acceptable salt, solvate, hydrate, or prodrug thereof. In some embodiments of the methods provided herein, the compound of Formula (I) is Compound A66 or an isotopic variant, pharmaceutically acceptable salt, solvate, hydrate, or prodrug thereof. In some embodiments of the methods provided herein, the compound of Formula (I) is Compound A67 or an isotopic variant, pharmaceutically acceptable salt, solvate, hydrate, or prodrug thereof.

In some embodiments, the B cell malignancy is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitts lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In certain embodiments, the B cell malignancy is selected from non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). In certain embodiments, the B cell malignancy is diffuse large B-cell lymphoma (DLBCL). In certain embodiments, the B cell malignancy is diffuse large B-cell lymphoma (DLBCL). In certain embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In some embodiments, the B cell malignancy is B-cell non-Hodgkin's lymphoma (NHL). In certain embodiments, the B-cell malignancy is selected from chronic lymphocytic leukemia (CLL), small lymphocytic lymphoma (SLL), follicular lymphoma (FL), marginal zone B cell lymphoma (MZL), diffuse large B-cell lymphoma (DLBCL), and high grade non-Hodgkin's lymphoma. In certain embodiments, the B-cell malignancy is selected from chronic lymphocytic leukemia (CLL), follicular lymphoma (FL), marginal zone B cell lymphoma (MZL), or diffuse large B-cell lymphoma (DLBCL). In certain embodiments, the B cell malignancy is a relapsed or refractory B cell malignancy. In certain embodiments of the methods provided herein, the FL is a relapsed or refractory FL (R/R FL). In certain embodiments of the methods provided herein, the FL is relapsed/refractory FL after failure of at least two prior lines of systemic therapy in the subject. In some embodiments of the methods provided herein, the FL is relapsed/refractory FL after failure of at least two prior lines of systemic therapy in the subject, wherein the systemic therapy comprises an anti-CD20 antibody and/or chemotherapy with an alkylating agent or a purine analog.

In some embodiments provided herein are methods of treating follicular lymphoma (FL) in a subject in need thereof, wherein the subject has failed two or more prior chemotherapies. In some embodiments provided herein are methods of treating follicular lymphoma (FL) in a subject in need thereof, wherein the subject has failed two or more prior systemic chemotherapies. In some embodiments provided herein are methods of treating follicular lymphoma (FL) in a subject in need thereof, wherein the subject has failed two or more prior systemic chemotherapies, wherein each systemic chemotherapy is selected from the group consisting of an anti-CD20 antibody, an alkylating chemotherapeutic agent, and a chemotherapeutic purine analog.

In some embodiments provided herein are methods comprising administering to a subject in need thereof a single pharmaceutical composition consisting of a compound of Formula (I), or an enantiomer, a mixture of enantiomers, a mixture of two or more diastereomers, or an isotopic variant thereof; or a pharmaceutically acceptable salt, solvate, or hydrate as monotherapy. In some embodiments, provided herein is a method of treating follicular lymphoma (FL), the method comprising administering to a subject in need thereof a therapeutically effective amount of a single pharmaceutical composition consisting of: (i) compound of Formula (I), or an enantiomer, a mixture of enantiomers, a mixture of two or more diastereomers, or an isotopic variant thereof; or a pharmaceutically acceptable salt, solvate, or hydrate; and (ii) one or more pharmaceutically acceptable carriers. Some embodiments provided herein describe a method of treating relapsed follicular lymphoma (FL), the method comprising administering to a subject in need thereof a therapeutically effective amount of a compound of Formula (I), or an enantiomer, a mixture of enantiomers, a mixture of two or more diastereomers, or an isotopic variant thereof; or a pharmaceutically acceptable salt, solvate, or hydrate as monotherapy. In some embodiments, provided herein is a method of treating relapsed follicular lymphoma (FL), the method comprising administering to a subject in need thereof a therapeutically effective amount of a single pharmaceutical composition consisting of: (i) compound of Formula (I), or an enantiomer, a mixture of enantiomers, a mixture of two or more diastereomers, or an isotopic variant thereof; or a pharmaceutically acceptable salt, solvate, or hydrate; and (ii) one or more pharmaceutically acceptable carriers.

### Dosages and Dosing Regimens

In some embodiments, the methods provided herein comprise administering a compound of Formula (I), or an isotopic variant thereof; or a pharmaceutically acceptable salt, solvate, hydrate, or prodrug thereof to a patient in need thereof.

In some instances, the method for multiple cycle chemotherapy comprises the administration of a second cycle within about 60 days or about 3 months. In some instances, the method for multiple cycle chemotherapy comprises the administration of a second cycle within 50 days. In another instance, the second cycle is administered within 45, 40, 35, 30, 25, 21, 20, 15, 14, 10, 9, 8, 7, 6, 5, 4, 3, 2 or 1 day(s) of the first cycle. In some embodiments, the administration of any additional cycles is within 50 days of the previous cycle. In some embodiments, the administration of any additional cycles is within 10 days of the previous cycle. In some embodiments, the administration of any additional cycles is within 9 days of the previous cycle. In some embodiments, the administration of any additional cycles is within 8 days of the previous cycle. In some embodiments, the administration of any additional cycles is within 7 days of the previous cycle. In some embodiments, the administration of any additional cycles is within 6 days of the previous cycle. In some embodiments, the administration of any additional cycles is within 5 days of the previous cycle. In some embodiments, the administration of any additional cycles is within 4 days of the previous cycle. In some embodiments, the administration of any additional cycles is within 3 days of the previous cycle. In some embodiments, the administration of any additional cycles is within 2 days of the previous cycle. In some embodiments, the administration of any additional cycles is within 1 day of the previous cycle. In another embodiment, the additional cycle is administered within 45, 40, 35, 30, 25, 21, 20, 15, 14, 10, 9, 8, 7, 6, 5, 4, 3, 2, or 1 days of the previous cycle.

The length of a treatment cycle depends on the treatment being given. In some embodiments, the length of a treatment cycle ranges from two to six weeks. In some embodiments, the length of a treatment cycle ranges from four to six weeks. In some embodiments, the length of a treatment cycle is 28 days. In some embodiments, the length of a treatment cycle is 56 days. In some embodiments, a treatment cycle lasts one, two, three, or four weeks. In some embodiments, a treatment cycle lasts four weeks. The number of treatment doses scheduled within each cycle also varies depending on the drugs being given.

In certain instances, the compound of Formula (I), or an isotopic variant thereof; or a pharmaceutically acceptable salt, solvate, hydrate, or prodrug thereof, is administered to the subject on a 28-day cycle. In some embodiments, the compound of Formula (I), or an isotopic variant thereof; or a pharmaceutically acceptable salt, solvate, hydrate, or prodrug thereof, is administered to the subject for at least one 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two 28-day cycles. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered daily to the subject on a 28-day continuous schedule until disease progression or intolerable toxicity.

In certain embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for a period of up to about 7 days. In some embodiments, the days over which the compound of Formula (I), or an enantiomer, a mixture of enantiomers, a mixture of two or more diastereomers, or an isotopic variant thereof; or a pharmaceutically acceptable salt, solvate, hydrate, or prodrug thereof are intermittent. In some embodiments, administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof for about 7 consecutive days in a 28-day cycle.

In some embodiments, the method comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle. In some embodiments, the IS avoids or reduces adverse or unwanted side effects associated with the use of the PI3K inhibitor, such as enterocolitis (manifested as diarrhea), cutaneous toxicities, liver toxicity (manifested as elevation of transaminases), pulmonary toxicity (manifested as non-infectious pneumonitis), and infections. In some embodiments, the IS avoids or reduces enterocolitis, rash, transaminitis, or combinations thereof.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for 28 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on continuous dosing schedule (CS). In some embodiments, the continuous dosing schedule (CS), comprises once daily administration of compound of Formula (I), or an enantiomer, a mixture of enantiomers, a mixture of two or more diastereomers, or an isotopic variant thereof; or a pharmaceutically acceptable salt, solvate, hydrate, or prodrug thereof to the subject for 28 consecutive days in a 28-day cycle. In some embodiments, the continuous dosing schedule (CS), comprises once daily administration of compound of Formula (I), or an enantiomer, a mixture of enantiomers, a mixture of two or more diastereomers, or an isotopic variant thereof; or a pharmaceutically acceptable salt, solvate, hydrate, or prodrug thereof to the subject for 28 consecutive days in a 28-day cycle until disease progression or intolerable toxicity. In some instances, patients on CS report of delayed onset of cases of enterocolitis and rash.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 intermittent days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of up to about 7 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of 7 consecutive days in a 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS). In some embodiments, the intermittent dosing schedule (IS), comprises once daily administration of compound of Formula (I), or an enantiomer, a mixture of enantiomers, a mixture of two or more diastereomers, or an isotopic variant thereof; or a pharmaceutically acceptable salt, solvate, hydrate, or prodrug thereof to the subject for 7 consecutive days followed by 21 days without treatment in a 28-day cycle.

In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three 28-day cycles, wherein: the first two 28-day cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for two 28-day cycles; and the third28-day cyclecomprises an intermittent dosing schedule (IS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for the first 7 consecutive days of the 28-day cycle. In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least three cycles, wherein: the first two cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for two cycles; and the subsequent cycle(s) comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for only the first 7 consecutive days in each subsequent cycle. In some embodiments of the methods provided herein, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for four or more 28-day cycles, wherein: the first two or three 28-day cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for three or more 28-day cycles; and the subsequent 28-day cycle(s) comprise(s) an intermittent dosing schedule (IS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof, once daily for the first 7 consecutive days of the 28-day cycle.

In certain instances, CS refers to continuous daily dosing to a subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily on a 28-day schedule with no switch to IS. In certain instances, CS refers to continuous daily dosing to a subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily on a 28-day schedule for four or more cycles followed by a switch to IS (i.e., late switch to IS). In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof is administered to a subject on an intermittent dosing schedule (IS) until progression of disease. In some embodiments, upon progression of disease, the subject resumes continuous daily dosing (CS) of the compound of Formula (I), or an enantiomer, a mixture of enantiomers, a mixture of two or more diastereomers, or an isotopic variant thereof; or a pharmaceutically acceptable salt, solvate, hydrate, or prodrug thereof.

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two cycles of continuous daily administration (CS) followed by daily administration for only the first seven days of each subsequent cycle, the CS and IS cycles are 28-day cycles. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) to reduce or mitigate adverse side effects associated with PI3K5 inhibitors (e.g., enterocolitis, rash, and/or transaminitis). In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in mitigation or reduction of the incidence of immune-mediated toxicities by allowing recovery of TREG during treatment-free intervals.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in disease stabilization. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in disease regression. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) resulting in an objective response. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease stabilization is no longer observed. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease progression is observed.

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two cycles of continuous daily administration (CS) followed by daily administration for only the first seven days of each subsequent (IS) cycle, the CS and IS cycles are 28-day cycles, wherein the IS cycle is repeated until disease regression is no longer observed. In some or additional embodiments, if disease progression is observed in the subject, the subject resumes the 28-day cycles of continuous daily administration (CS) until disease regression or stabilization are observed

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two 28-day cycles of continuous daily administration (CS) followed by daily administration for only the first seven days of each subsequent (IS) 28-day cycle; wherein disease regression or stabilization is no longer observed in the subject on the intermittent dosing schedule (IS) cycle, the subject resumes 28-day cycles of continuous daily administration (CS) until disease regression or stabilization are observed.

In some embodiments, about 60 mg of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of 7 consecutive days in a 28-day cycle followed by 21 days without therapy, with cycles repeated every 28 days.

In some embodiments, administration of about 60 mg of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to a subject in need thereof once daily for a period of 7 consecutive days followed by 21 days without treatment in a 28-day cycle results in steady-state plasma concentrations sufficient to inhibit PI3K5 in target malignant B-cells. In further or additional embodiments, the subsequent 21 days without treatment is sufficient to repopulate TREG (i.e., 7 days to clear the compound of Formula (I) from the plasma (˜7 half-lives) and 14 days for reconstitution of TREG after the compound of Formula (I) is cleared from the plasma.

In certain instances, the method comprises a continuous daily dosing schedule (CS) for at least two CS 28-day cycles, followed by an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle after the at least two CS 28-day cycles. In some embodiments, the dosing schedule avoids or reduces adverse or unwanted side effects associated with the use of the PI3K inhibitor, such as enterocolitis (manifested as diarrhea), cutaneous toxicities, liver toxicity (manifested as elevation of transaminases), pulmonary toxicity (manifested as non-infectious pneumonitis), and infections. In some embodiments, the dosing schedule avoids or reduces enterocolitis, rash, transaminitis, or combinations thereof.

In some instances, the method for the administration of multiple compounds comprises administering compounds within 48 hours or less of each other. In some embodiments administration occurs within 24 hours, 12 hours, 6 hours, 3 hours, 1 hour, or 15 minutes. In some instances, the compounds are administered simultaneously. One example of simultaneous administration is the injection of one compound immediately before, after, or during the oral administration of the second compound, immediately referring to a time less than about 5 minutes.

In some embodiments, the compound of Formula (I), or an isotopic variant thereof; or a pharmaceutically acceptable salt, solvate, hydrate, or prodrug thereof, is administered in an amount of about 30, about 60 mg, about 120 mg, about 150 mg, or about 180 mg. In certain embodiments, the compound of Formula (I), or an isotopic variant thereof; or a pharmaceutically acceptable salt, solvate, hydrate, or prodrug thereof is administered in an amount of about 60 mg. In certain embodiments, the compound of Formula (I), or an isotopic variant thereof; or a pharmaceutically acceptable salt, solvate, hydrate, or prodrug thereof is administered in an amount of about 30 mg/day. In certain embodiments, the compound of Formula (I), or an isotopic variant thereof; or a pharmaceutically acceptable salt, solvate, hydrate, or prodrug thereof is administered in an amount of about 45 mg/day. In certain embodiments, the compound of Formula (I), or an isotopic variant thereof; or a pharmaceutically acceptable salt, solvate, hydrate, or prodrug thereof is administered in an amount of about 60 mg/day. In certain embodiments, the compound of Formula (I), or an isotopic variant thereof; or a pharmaceutically acceptable salt, solvate, hydrate, or prodrug thereof is administered in an amount of about 90 mg/day. In certain embodiments, the compound of Formula (I), or an isotopic variant thereof; or a pharmaceutically acceptable salt, solvate, hydrate, or prodrug thereof is administered in an amount of about 120 mg/day. In certain embodiments, the compound of Formula (I), or an isotopic variant thereof; or a pharmaceutically acceptable salt, solvate, hydrate, or prodrug thereof is administered in an amount of about 150 mg/day. In certain embodiments, the compound of Formula (I), or an isotopic variant thereof; or a pharmaceutically acceptable salt, solvate, hydrate, or prodrug thereof is administered in an amount of about 180 mg/day.

For oral administration, the pharmaceutical compositions provided herein can be formulated in the form of tablets containing from about 1.0 to about 1,000 mg of a compound of Formula (I), or an isotopic variant thereof; or a pharmaceutically acceptable salt, solvate, hydrate, or prodrug thereof, in one embodiment, about 1, about 5, about 10, about 15, about 20, about 25, about 30, about 50, about 60, about 75, about 100, about 120, about 150, about 180, about 200, about 250, about 300, about 400, about 500, about 600, about 750, about 800, about 900, and about 1,000 mg of the a compound of Formula (I), or an isotopic variant thereof; or a pharmaceutically acceptable salt, solvate, hydrate, or prodrug thereof for the symptomatic adjustment of the dosage to the patient to be treated.

In some embodiments, the pharmaceutical compositions provided herein can be formulated in the form of tablets containing about 30 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 30 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 30 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 30 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 30 mg daily until disease progression or intolerable toxicity.

In some embodiments, the pharmaceutical compositions provided herein can be formulated in the form of tablets containing about 45 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 45 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 45 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 45 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 45 mg daily until disease progression or intolerable toxicity.

In some embodiments, the pharmaceutical compositions provided herein can be formulated in the form of tablets containing about 60 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 60 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 60 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 60 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 60 mg daily until disease progression or intolerable toxicity.

In some embodiments, the pharmaceutical compositions provided herein can be formulated in the form of tablets containing about 90 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 90 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 90 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 90 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 90 mg daily until disease progression or intolerable toxicity.

In some embodiments, the pharmaceutical compositions provided herein can be formulated in the form of tablets containing about 120 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 120 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 120 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 120 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 120 mg daily until disease progression or intolerable toxicity.

In some embodiments, the pharmaceutical compositions provided herein can be formulated in the form of tablets containing about 150 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 150 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 150 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 150 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 150 mg daily until disease progression or intolerable toxicity.

In some embodiments, the pharmaceutical compositions provided herein can be formulated in the form of tablets containing about 180 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 180 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 180 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 180 mg daily for 56 days. In certain embodiments, a compound of Formula (I), or an isotopic variant thereof; or a pharmaceutically acceptable salt, solvate, hydrate, or prodrug thereof is administered to a patient in need thereof in an amount of about 180 mg daily until disease progression or intolerable toxicity.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Articles of Manufacture

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes one or more containers and a dosage form of a compound of Formula (I), or an isotopic variant thereof; or a pharmaceutically acceptable salt, solvate, hydrate, or prodrug thereof.

In certain embodiments, the kit provided herein includes one or more containers and a dosage form of a compound of Formula (I), or an isotopic variant thereof; or a pharmaceutically acceptable salt, solvate, hydrate, or prodrug thereof. Kits provided herein can further include devices that are used to administer the active ingredients. Examples of such devices include, but are not limited to, syringes, needle-less injectors drip bags, patches, and inhalers.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL, (microliters); M (molar); mM (millimolar), μM (micro molar); eq. (equivalent); mmol (millimoles), Hz (Hertz), MHz (megahertz); hr or hrs (hour or hours); min (minutes); and MS (mass spectrometry).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated herein are intended to exemplify the applicable chemistry through the use of specific examples and are not indicative of the scope of the disclosure.

Synthesis of any of the Formulae provided herein e.g., Formulae (I), (II), (VII), (IX), (X), (XI), (XVI), is described in U.S. Pat. No. 9,056,852 B2, which is incorporated by reference for such disclosure.

### Example 1

In some instances, severe cases of enterocolitis, rash, and transaminitis have been reported in a Phase 1b clinical study of Compound A35 in patients with B-cell malignancies. In some instances, the onset of severe immune-related toxicities were reported after a period of CS dosing greater than 2 cycles. A lymphocytic infiltrate was reported in biopsies obtained from 1 patient with colitis and 1 patient with severe skin rash enrolled in the study. Furthermore, corticosteroid therapy has been reported as effective treatment approach in patients who developed diarrhea and rash in the study.

### Example 2: Study of Treatment of Relapsed B-Cell Malignancies

Following reports of delayed onset of cases of enterocolitis and rash in patients with Compound A35 initially administered once a day on a continuous dosing schedule (CS) in a 28-day cycle, an alternate dosing regimen using an intermittent dosing schedule (IS) was evaluated. The hypothesis was that an IS of PI3K5 inhibitors including Compound A35 could reduce the incidence or lower the severity of immune mediated adverse events (irAEs) by allowing recovery of TREGs during treatment-free intervals. Furthermore, it was hypothesized that the IS, by delivering a lower dose-intensity treatment approach, could be used to re-treat patients who have experienced delayed irAEs on the CS or IS and had recovered following treatment interruption and a short course of corticosteroids.

Parsaclisib, using a day per week dosing after an initial once daily dosing for two 28-day cycles, was evaluated on IS. Preliminary data indicated a decrease in the incidence of severe irAEs, but a high rate of tumor progression on IS. The rationally-designed IS in this study consists of Compound A35 administered once a day on 7 consecutive days followed by 21 days without therapy, with cycles repeated every 28 days. This schedule was based on the known plasma half-life of Compound A35 and the kinetics of TREGs repopulation. (FIGS. 6A-6B.)

In healthy volunteers and in patients with B-cell malignancies, Compound A35 has a demonstrated plasma half-life of approximately 28 hours. It is known that administration of an oral drug over a period equivalent to approximately 7 half-lives is necessary to achieve steady state plasma concentrations required for optimal antitumor activity, and that a period of approximately 7 half-lives is needed to clear a drug from the plasma after treatment interruption. TREGs repopulation occurs approximately 14 days after the administration of a single dose of the anti-CD25 immunotoxin denileukin diftitox (ONTAK®), a drug known to suppress TREGs. Therefore, it was hypothesized that administration of Compound A35 at 60 mg/day for 7 consecutive days will result in steady-state plasma concentrations sufficient to inhibit PI3K5 in target malignant B-cells and that 21 days without treatment will be sufficient to repopulate TREGs, which comprises 7 days to clear Compound A35 from the plasma (˜7 half-lives) and 14 days for reconstitution of TREGs after Compound A35 is cleared from the plasma.

Furthermore, since the onset of severe irAEs were delayed in patients treated with Compound A35 initially administered once a day on a continuous dosing schedule (CS) in a 28-day cycle, which was typically reported after a period of CS dosing greater than 2 cycles, the goal was to begin the IS after 2 cycles of CS. The 2 cycles of CS serve as tumor debulking and the subsequent cycles of IS are for maintaining disease control. Preliminary data provides preliminary evidence this IS schedule was successful in reducing the risk of toxicities without erosion in treatment efficacy in most patients.

The analysis presented here is limited to patients with the indolent B-cell malignancies FL and CLL, and its variant small lymphocytic lymphoma (SLL) as they represent a homogeneous group of patients.

Preliminary data suggest that the rationally developed IS based on the known mechanism of action of Compound A35, its plasma concentration half-life, and TREG repopulation kinetics, appears to reduce the incidence of severe irAEs without erosion in treatment efficacy in most patients.

Intermittent Dosing Schedule demonstrates improved tolerability compared to continuous dosing schedule.

An intermittent dosing schedule on days 1-7 of a 28-day cycle was then evaluated after 2 cycles of daily dosing, or ≥3 cycles of daily dosing to reduce the risk of immune-related adverse events. For this analysis the intermittent schedule (IS) group is defined as patients who received Compound A35 alone or with rituximab daily for 2 cycles then switched to an intermittent schedule of 1 week per cycle, and the continuous schedule (CS) group is defined as patients who never switched to intermittent dosing or switched to intermittent dosing in cycle 4 or later cycles. Toxicity on CS is managed by a switch to IS.

Progression of disease on IS is managed by a switch to CS.

In an ongoing Phase 1b study, Compound A35 was administered as a single agent in patients with relapsed B-cell malignancies. Patients have been enrolled in 3 cohorts:
Group A: 31 patients enrolled in a dose escalation phase of the study, with Compound A35 administered as a single agent at a dose of 60, 120, and 180 mg/day. In Group A, all patients began receiving Compound A35 on a CS. Approximately 1.5 years after the start of the study, 17 ongoing patients were switched to the IS to prevent delayed immune-mediated toxicities after various duration of exposure on the CS. The demographics and disease characteristics in the dose escalation cohort are shown in Table 1.

TABLE 1
Baseline Characteristics in Dose Escalation Cohort

| | FL | | CLL/SLL | | Total | |
|---|---|---|---|---|---|---|
| | N = 22 | | N = 9 | | N = 31 | |
| Age in years, median (range) | 65 | (47-76) | 60 | (50-79) | 65 | (47-79) |
| Men, N (%) | 14 | (64%) | 7 | (78%) | 21 | (68%) |
| Number of prior therapies, median (range) | 2 | (1-5) | 1 | (1-2) | 1 | (1-5) |
| Subjects with prior anti-CD20 therapy, N (%) | 22 | (100%) | 7 | (78%) | 29 | (94%) |
| Subjects with prior alkylating therapy, N (%) | 19 | (86%) | 8 | (89%) | 27 | (87%) |
| Subjects with lymph nodes ≥5 cm, N (%) | 11 | (50%) | 5 | (56%) | 16 | (52%) |

Group B: 21 patients enrolled in an ongoing expansion cohort of up to 30 patients administered Compound A35 as a single agent at 60 mg/day on the CS for 2 cycles then switching to the IS in Cycle 3 (N=17) or later cycles (N=4).

Group A: Monotherapy on the CS with Later Switch to the IS

31 patients received Compound A35 monotherapy at doses ≥60 mg daily on the CS, of whom 2 discontinued Compound A35 in the first 2 cycles of therapy and 29 received >2 cycles.

A very high rate of disease response was reported in 30 patients evaluable for response in the dose escalation cohort, both in FL and CLL/SLL, with no difference in response rate across the 3 doses evaluated (Table 2).

TABLE 2
Response Rate with Compound A35 Alone

| | 60 mg | | 120 mg | | 180 mg | | Total | |
|---|---|---|---|---|---|---|---|---|
| | N = 12 | | N = 12 | | N = 6 | | N = 30 | |
| FL (N = 21 evaluable) | n = 6 | | n = 10 | | n = 5 | | n = 21 | |
| Overall response rate | 5 | (83%) | 9 | (90%) | 4 | (80%) | 18 | (86%) |
| Morphologic and/or metabolic complete response | 2 | (33%) | 4 | (40%) | 0 | | 6 | (21%) |
| CLL/SLL (N = 9) | n = 6 | | n = 2 | | n = 1 | | n = 9 | |
| Overall response rate | 6 | (100%) | 2 | (100%) | 1 | (100%) | 9 | (100%) |
| Morphologic complete response | 3 | (50%) | 0 | | 0 | | 3 | (33%) |
| All evaluable patients (N = 30) | n = 12 | | n = 12 | | n = 6 | | n = 30 | |
| Overall response rate | 11 | (92%) | 11 | (92%) | 5 | (83%) | 27 | (90%) |

| | | | | | | | |
|---|---|---|---|---|---|---|---|
| Morphologic and/or metabolic complete response | 5 | (42%) | 4 | (33%) | 0 | 9 | (30%) |

TABLE 3
Response Rate with Compound A35 Alone
in Patients Evaluable for Response*

| | FL | CLL/SLL | Total |
|---|---|---|---|
| Patient Groups | N = 38 | N = 11 | N = 49 |
| Compound A35 alone | 30/38 (79%) | 11/11 (100%) | 41/49 (84%) |
| on IS or CS | | | |
| IS Group | 9/14 (64%) | 2/2 (100%) | 11/16 (69%) |
| CS Group | 21/24 (88%) | 9/9 (100%) | 30/33 (91%) |

*At least one post baseline assessment

Aggregating the results for Groups A and B shows that Compound A35 alone achieves a high response rate both in relapsed FL and CLL/SLL, and whether administered on IS or CS (Table 3).

The IS was developed to mitigate the risk of delayed toxicities observed with PI3K inhibitors and believed to be associated with on-target effect PI3K inhibition on immune cells. The incidence of severe adverse events of special interest (AESI) associated with PI3K inhibition was substantially reduced with the IS dosing compared to the CS dosing (Tables 4 and 5).

TABLE 4
Adverse Events of Special Interest with Compound A35 Administered by IS or CS for FL

| | All Grades | | | Related Grade 3 | | |
|---|---|---|---|---|---|---|
| | All | CS Group | IS Group | All | CS Group | IS Group |
| AESI | (N = 40) | (N = 25) | (N = 15) | (N = 40) | (N = 25) | (N = 15) |
| Diarrhea/ | 17 (42.5%) | 10 (40.0%) | 7 (46.7%)* | 6 (15.0%) | 5 (20.0%) | 1 (6.7%) |
| colitis | | | | | | |
| Rash, all | 12 (30.0%) | 11 (44.0%) | 1 (6.7%) | 4 (10.0%) | 4 (16.0%) | 0 |
| types | | | | | | |
| ALT/AST | 9 (22.5%) | 6 (24.0%) | 3 (20.0%) | 3 (7.5%) | 3 (12.0%) | 0 |
| increased | | | | | | |
| Pneumonia/ | 2 (5.0%) | 2 (8.0%) | 0 | 2 (5.0%) | 2 (8.0%) | 0 |
| Pneumonitis | | | | | | |
| Mucositis | 6 (15.0%) | 6 (24.0%) | 0 | 1 (2.5%) | 1 (4.0%) | 0 |

*Excluded 1 subject who had diarrhea after POD, and 1 subject who had diarrhea which only lasted one day with no dose change

TABLE 5
Adverse Events of Special Interest with Compound
A35 Administered by IS or CS for CLL/SLL

| | All Grades | | | Related Grade 3 | | |
|---|---|---|---|---|---|---|
| | All | CS Group | IS Group | All | CS Group | IS Group |
| AESI | (N = 17) | (N = 9) | (N = 8) | (N = 17) | (N = 9) | (N = 8) |
| Diarrhea/ | 9 (52.9%) | 6 (66.7%) | 3 (37.5%) | 4 (23.5%) | 3 (33.3%) | 1 (12.5%) |
| colitis | | | | | | |
| Rash, all | 6 (35.3%) | 4 (44.0%) | 2 (25.0%) | 0 | 0 | 0 |
| types | | | | | | |
| ALT/AST | 2 (11.8%) | 2 (22.2%) | 0 | 0 | 0 | 0 |
| increased | | | | | | |
| Pneumonia/ | 3 (17.6%) | 3 (33.3%) | 0 | 3 (17.6%) | 3 (33.3%) | 0 |
| Pneumonitis | | | | | | |
| Mucositis | 1 (5.9%) | 1 (11.1%) | 0 | 0 | 0 | 0 |

Therefore, data suggest that the IS administration of compound A35 reduces the incidence of or delayed onset of immune-mediated toxicities without erosion in treatment efficacy.

TABLE 6
Patient Dispositions on Intermittent or Continuous
Schedule in Compound A35 Alone Arm

| | | |
|---|---|---|
| | CS Group | IS Group |
| Patient Disposition | (N = 25) | (N = 15) |

| Continuing on therapy | 10 | (40%) | 11 | (73%) |
|---|---|---|---|---|
| Discontinued therapy | | | | |
| Progressive disease | 5 | (20%) | 3 | (20%) |
| Adverse event | 4 | (16%) | 0 | |
| Stem cell transplant | 3 | (12%) | 1 | (7%) |
| Withdrawal of consent | 3 | (12%) | 0 | |
| Follow-up (months) | | | | |
| Median (range) | 6.0 | (0.9-25.8) | 5.5 | (0.9-15.5) |

### Example 3: Phase 2 Study of Compound A35 in Subjects with Follicular Lymphoma after Failure of Two or More Prior Systemic Therapies

Patients will be enrolled in a global randomized, double blind, placebo-controlled study to compare the efficacy and safety of Compound A35 administered on the CS or the CS×2 cycles then the IS in a large cohort of patients with relapsed FL. A correlative immune study is performed on a subset of patients enrolled in the study to evaluate the effect of Compound A35 on T-cell subsets, including TREG, and any possible association between TREG and delayed immune-related toxicities.

Brief Summary: This is the study of the PI3K5 inhibitor Compound A35 in subjects with relapsed/refractory Follicular Lymphoma (FL) after failure of at least 2 prior lines of systemic therapy

Detailed Description: This is a global, multicenter, randomized, double-blind, placebo-controlled, 2 arm, Phase 2 study of the PI3K5 inhibitor Compound A35 in subjects with relapsed/refractory Follicular Lymphoma after failure of at least 2 prior lines of systemic therapy which must have included an antiCD20 antibody and chemotherapy with an alkylating agent or a purine analog. The study will evaluate the efficacy and safety of Compound A35 administered using two different schedules: daily continuously or daily continuously for 2 cycles then daily for the first 7 days of each subsequent cycle. Approximately 165 subjects will be randomized into the study.

Study Design: Two-Arm, Phase 2 Study

### Allocation: Randomized

### Intervention Model: Parallel Assignment

### Masking: Quadruple (Participant, Care Provider, Investigator, Outcomes Assessor)

Arms and Interventions

Compound A35 is provided as a capsule, taken orally.
Group A: Continuous schedule. Compound A35 is administered daily continuously. Compound A35 is a capsule, taken orally once a day (60 mg/day).
Group B: Intermittent schedule. Compound A35 is administered daily continuously for 2 cycles then daily for the first 7 days of each subsequent cycle.

Primary Outcome Measure:

1. Objective response rate (ORR) [Time Frame: 2 years]
ORR of Compound A35 in relapsed Follicular Lymphoma, defined as the best response rating of complete response (CR) or partial response (PR) according to the Lugano Response Criteria (Cheson 2014), as determined by an Independent Response Review Committee (IRRC)
2. Tolerability of Compound A35 [Time Frame: 2 years]
Tolerability of Compound A35, defined as the rate of AEs requiring modified dosing schedule or study drug discontinuation (AERDM)

Secondary Outcome Measures:

1. Efficacy of Compound A35 as assessed by an IRRC [Time Frame: 2 years]
a. Duration of response (DOR) among subjects with an objective response
b. Complete response (CR) rate
c. Progression-free survival (PFS)
   2. Efficacy of Compound A35 as assessed by the Investigator [Time Frame: 2 years]
a. Objective response rate (ORR)
b. Duration of response (DOR) among subjects with an objective response
c. Complete response (CR) rate
d. Progression-free survival (PFS)
   3. Overall survival (OS) [Time Frame: 2 year]
   Overall survival
   4. Safety profile of Compound A35 [Time Frame: 2 years]
   Overall incidence of AEs and Time to occurrence of AERDM
   5. To evaluate the PK of Compound A35 [Time Frame: 6 months] PK evaluation

Eligibility Criteria

Ages Eligible for Study: 18 Years and older

### Sexes Eligible for Study: All

### Gender Based: No

### Accepts Healthy Volunteers: No

Inclusion Criteria:

1. Histologically confirmed diagnosis of FL as defined in the World Health Organization (WHO) classification scheme, limited to Grade 1, 2, or 3a
2. Progression of disease after at least 2 prior systemic therapies for FL
3. No prior therapy with PI3K5 inhibitors
4. No disease progression on prior therapy with Bruton tyrosine kinase (BTK) inhibitors
5. At least one bi-dimensionally measurable nodal lesion >1.5 cm in its longest diameter by computed tomography (CT) scan as defined by the Lugano Classification
6. Adequate hematologic, renal and hepatic parameters at screening unless abnormal values are due to FL per Investigator assessment
7. QT-interval corrected according to Fridericia's formula (QTcF)<450 milliseconds (msec);
8. Left ventricular ejection fraction (LVEF)≥than institutional lower limit of normal as measured by echocardiogram

Exclusion Criteria:

1. Known active histological transformation from FL to an aggressive lymphoma
2. Any uncontrolled clinically significant illness
3. Subjects who have tested positive for hepatitis B surface antigen and/or hepatitis B core antibody plus have a positive hepatitis B
4. Ongoing or history of drug-induced pneumonitis
5. History of clinically significant cardiovascular abnormalities
6. History of clinically significant GI conditions

What is claimed is:

1. A method of treating cancer, comprising administering to a subject in need thereof a single pharmaceutical composition consisting of:
(i) about 30 mg, about 60 mg, about 120 mg, or about 180 mg of a compound of Formula (I): 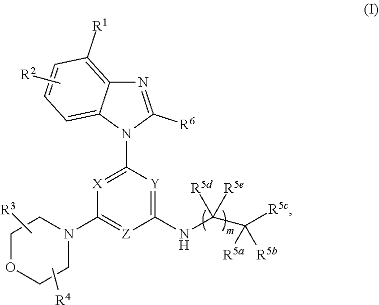
or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
—S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or
—S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or
—S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h},
—NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl; and
(ii) one or more pharmaceutically acceptable carriers.

2. The method of claim 1, wherein about 60 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

3. The method of claim 1 or 2, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject daily.

4. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day, twice per day, or three times per day.

5. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day.

6. The method of any one of the preceding claims, wherein about 60 mg/day of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

7. The method of any one of claims 1-6, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on a 28-day cycle.

8. The method of any one of claims 1-7, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle.

9. The method of any one of claims 1-8, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two 28-day cycles.

10. The method of any one of claims 1-9, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for a period of up to about 7 days.

11. The method of any one of claims 1-10, wherein the days over which the compound of Formula (I), or an enantiomer, a mixture of enantiomers, a mixture of two or more diastereomers, or an isotopic variant thereof; or a pharmaceutically acceptable salt, solvate, hydrate, or prodrug thereof are intermittent.

12. The method of any one of claims 1-11, comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof for about 7 consecutive days in a 28-day cycle.

13. The method of claim 1 or 12, wherein the method comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

14. The method of claim 13, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject until disease progression or intolerable toxicity.

15. The method of any one of claims 1-8, wherein the method comprises a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle.

16. The method of claim 15, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two CS 28-day cycles.

17. The method of claim 16, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once weekly after the at least two CS 28-day cycles until disease progression or intolerable toxicity.

18. The method of claim 17, further comprising an IS, comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle after the at least two CS 28-day cycles.

19. A method of treating cancer, comprising administering to a subject in need thereof a therapeutically effective amount of a compound of Formula (I): 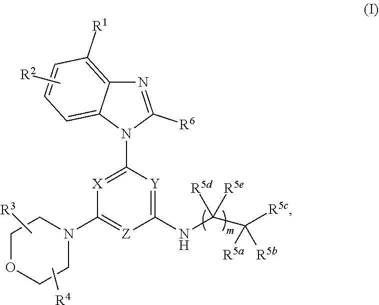
or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen;
(ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h},
—NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once daily for a period of about 7 days in a 28-day cycle.

20. The method of claim 19, wherein the days over which the compound of Formula (I), or an enantiomer, a mixture of enantiomers, a mixture of two or more diastereomers, or an isotopic variant thereof; or a pharmaceutically acceptable salt, solvate, hydrate, or prodrug thereof are intermittent.

21. The method of claim 19 or 20, comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof for about 7 consecutive days in a 28-day cycle.

22. The method of any one of claims 19-21, wherein the method comprises an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

23. The method of claim 22, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle.

24. A method of treating cancer, comprising administering to a subject in need thereof a therapeutically effective amount of a compound of Formula (I): 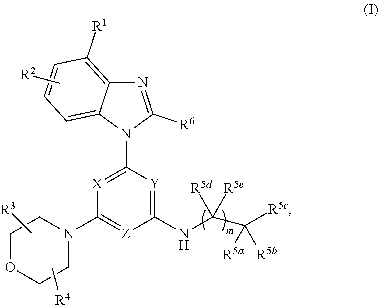
or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a}; wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl, wherein the method comprises at least three 28-day cycles,
wherein:
(i) the first two cycles comprise a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle; and
(ii) the third and subsequent cycles comprise an intermittent dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle.

25. The method of any one of claims 13, 14, or 22-24, wherein T-cells are recovered and/or re-populated during the 21 days without treatment.

26. The method of any one of claims 13, 14, or 22-25, wherein regulatory T-cells (TREG) and/or effector T-cells are recovered and/or re-populated during the 21 days without treatment.

27. The method of any one of claims 13, 14, or 22-26, wherein the incidence of at least one toxicity is reduced.

28. The method of claim 27, wherein the at least one toxicity is enterocolitis, a cutaneous toxicity, liver toxicity, pulmonary toxicity, infection, or any combination thereof.

29. The method of any one of claims 19-28, wherein about 60 mg of the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

30. The method of any one of the preceding claims, wherein the cancer is acute leukemia, acute lymphocytic leukemia, acute myelocytic leukemia, myelodysplastic syndrome, refractory anemia, refractory anemia with ringed sideroblasts, refractory anemia with excess blasts, refractory anemia with excess blasts in transformation, preleukemia, chronic myelomonocytic leukemia, chronic myelocytic (granulocytic) leukemia, chronic lymphocytic leukemia, hairy cell leukemia, polycythemia vera, Hodgkin's disease, non-Hodgkin's disease, multiple myeloma, Waldenstrom's macroglobulinemia, monoclonal gammopathy of undetermined significance, benign monoclonal gammopathy, heavy chain disease, bone and connective tissue sarcoma, brain tumor, breast cancer, adrenal cancer, thyroid cancer, pancreatic cancer, pituitary cancer, eye cancer, vaginal cancer, vulvar cancer, cervical cancer, uterine cancer, ovarian cancer, esophageal cancer, stomach cancer, colon cancer, rectal cancer, liver cancer, hepatocellular carcinoma, hepatoblastoma, gallbladder cancer, adenocarcinoma, cholangiocarcinoma, lung cancer, testicular cancer, prostate cancer, penal cancer, oral cancer, basal cancer, salivary gland cancer, pharynx cancer, skin cancer, kidney cancer, bladder cancer, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinoma.

31. The method of claim 30, wherein the cancer is leukemia, lymphoma, multiple myeloma, sarcoma, a brain tumor, breast cancer, adrenal cancer, thyroid cancer, pancreatic cancer, pituitary cancer, cervical cancer, ovarian cancer, esophageal cancer, stomach cancer, colon cancer, rectal cancer, liver cancer, lung cancer, testicular cancer, prostate cancer, or skin cancer.

32. The method of claim 30, wherein the cancer is acute leukemia, acute lymphocytic leukemia, acute myelocytic leukemia, chronic myelomonocytic leukemia (CMML), chronic myelocytic (granulocytic) leukemia, chronic lymphocytic leukemia, hairy cell leukemia, Hodgkin's disease, non-Hodgkin's disease, smoldering multiple myeloma, nonsecretory myeloma, osteosclerotic myeloma, plasma cell leukemia, solitary plasmacytoma, extramedullary plasmacytoma, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma, diffuse malignant lymphoma, non-small cell lung cancer, large-cell carcinoma, small-cell lung cancer, or basal cell carcinoma.

33. The method of claim 30, wherein the cancer is chronic lymphocytic leukemia or non-Hodgkin's lymphoma.

34. The method of claim 30, wherein the cancer is a hematological cancer or malignancy.

35. The method claim 30, wherein the cancer is a B-cell malignancy.

36. The method of claim 30, wherein the cancer is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

37. The method of any one of the preceding claims, wherein the cancer is non-Hodgkin's lymphoma diffuse large B-cell lymphoma (DLBCL).

38. The method of any one of the preceding claims, wherein the cancer is relapsed/refractory diffuse large B-cell lymphoma (r/r DLBCL).

39. The method of claim 37 or 38, wherein the diffuse large B-cell lymphoma is of the activated B-cell (ABC DLBCL) or Germinal center B-cell (GCB DLBCL).

40. The method of any one of claims 1-39, wherein the cancer is follicular lymphoma (FL).

41. The method of any one of claims 1-40, wherein the cancer is relapsed/refractory follicular lymphoma.

42. The method of any one of claims 1-41, wherein the cancer is relapsed/refractory follicular lymphoma after failure of at least two prior lines of systemic therapy in the subject.

43. The method claim 42, wherein the systemic therapy comprises an antiCD20 antibody and chemotherapy with an alkylating agent or a purine analog.

44. A method of treating follicular lymphoma (FL), comprising administering to a subject in need thereof a single pharmaceutical composition consisting of:
(i) a compound of Formula (I): 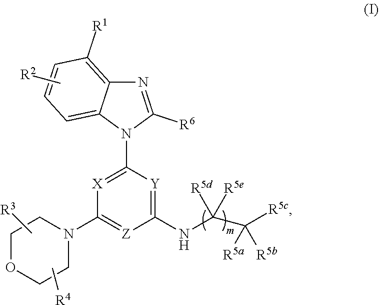
or an enantiomer, a mixture of enantiomers, a mixture of two or more diastereomers, or an isotopic variant thereof; or a pharmaceutically acceptable salt, solvate, or hydrate;
wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or
—S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, or four substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a}; wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl; and
(ii) one or more pharmaceutically acceptable carriers.

45. The method of claim 44, wherein the FL is relapsed/refractory FL.

46. The method of claim 45, wherein the cancer is relapsed/refractory FL after failure of at least two prior lines of systemic therapy in the subject.

47. The method claim 46, wherein the systemic therapy comprises an antiCD20 antibody and chemotherapy with an alkylating agent or a purine analog.

48. The method any one of claims 44-47, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject orally.

49. The method of any one of claims 44-48, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule.

50. The method of any one of claims 44-49, wherein about 30 mg, about 60 mg, about 120 mg, or about 180 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

51. The method of any one claims 44-50, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least one 28-day cycle.

52. The method of any one claims 44-50, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for two 28-day cycles.

53. The method of any one of claims 44-50, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered daily to the subject on a continuous schedule until disease progression or intolerable toxicity.

54. The method of claim 53, wherein the cycle is a 28-day cycle.

55. The method of any one of claims 44-50, wherein the method comprises at least three cycles, wherein:
(i) the first two cycles comprise a continuous daily dosing schedule (CS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for two cycles; and
(ii) the subsequent cycle(s) comprises an intermittent dosing schedule (IS), comprising administering to the subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for only the first 7 consecutive days in each subsequent cycle.

56. The method of claim 55, wherein the CS and IS cycles are each 28-day cycles.

57. The method of claim 55 or 56, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject on an intermittent dosing schedule (IS) until disease progression occurs.

58. The method of claim 57, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered daily to the subject on a continuous dosing schedule (CS) after disease progression occurs on an intermittent dosing schedule (IS).

59. The method of any one of claims 1-58, wherein R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

60. The method any one of claims 1-58, wherein R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

61. The method of claim 60, wherein R^{5a} and R^{5b} are each methyl, optionally substituted with one, two, or three halo(s).

62. The method of any one of claims 1-61, wherein n is 1.

63. The method of any one of the preceding claims, wherein R^{5f} and R^{5g} are each hydrogen.

64. The method of any one of claims 1-61, wherein n is 0.

65. The method of any one of the preceding claims, wherein m is 0.

66. The method of any one of the preceding claims, wherein the compound of Formula (I) is of Formula (XI): 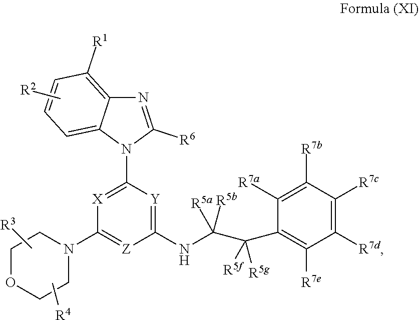
or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or
two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

67. The method of any one of claims 1, 19, 24, or 44,
wherein:
X, Y, and Z are each N;
R¹ and R² are each hydrogen;
R³ and R⁴ are each hydrogen;
R^{5a} is C₁₋₆ alkyl;
R^{5b} is C₁₋₆ alkyl;
R^{5c} is —(CH₂)-phenyl, wherein R^{5c} is optionally substituted with one, two, three, or four substituents Q;
R^{5d} and R^{5e} are each hydrogen;
R⁶ is CHF₂;
m is 0; and
wherein each alkyl is optionally substituted with one, two, three, or four substituents Q, wherein each substituent Q is independently selected from C₆₋₁₄ aryl, heteroaryl, and heterocyclyl, each of which is further optionally substituted with one, two, three, or four substituents Q^{a}, wherein the heteroaryl has from 5 to 10 ring atoms and one or more heteroatoms independently selected from O, S, and N, and the heterocyclyl has from 3 to 15 ring atoms and one or more heteroatoms independently selected from O, S, and N;
wherein each Q^{a} is independently selected from the group consisting of halo, C₁₋₆ alkyl, C₁₋₆ alkylsulfonyl and —OR^{e}, wherein R^{e} is hydrogen or C₁₋₆ alkyl.

68. The method of claim 67, wherein R^{5a} and R^{5b} are each methyl, optionally substituted with one or more halo.

69. The method of any one of claims 1-68, wherein the compound of Formula (I) is Compound A35: 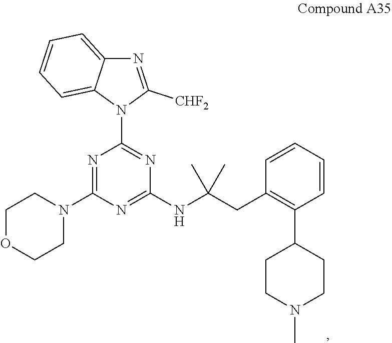 [SMILES: CN1CCC(c2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

70. The method of any one of claims 1-68, wherein the compound of Formula (I) is Compound A36: 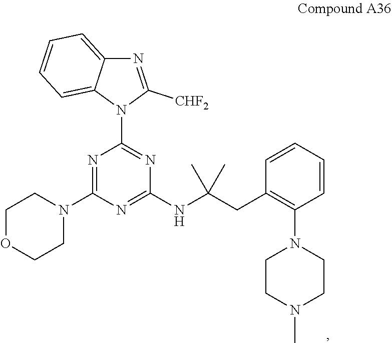 [SMILES: CN1CCN(c2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

71. The method of any one of claims 1-68, wherein the compound of Formula (I) is Compound A68: 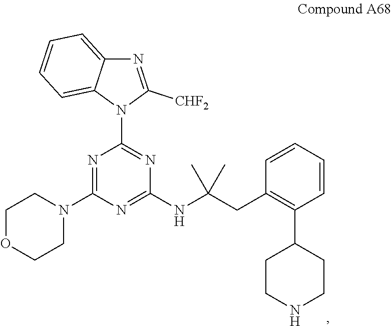 [SMILES: CC(C)(Cc1ccccc1C1CCNCC1)Nc1nc(N2CCOCC2)nc(-n2c(C(F)F)nc3ccccc32)n1]
or an isotopic variant thereof, a pharmaceutically acceptable salt, solvate, hydrate, or prodrug thereof.

72. The method of any one of claims 1-68, wherein the compound of Formula (I) is Compound A70: 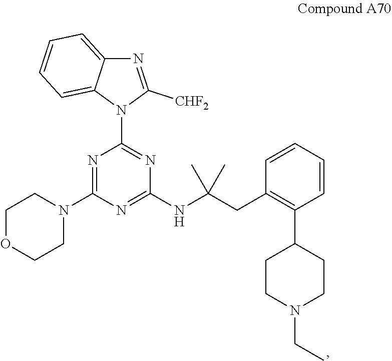 [SMILES: CCN1CCC(c2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

73. The method of any one of claims 1-68, wherein the compound of Formula (I) is Compound A37: 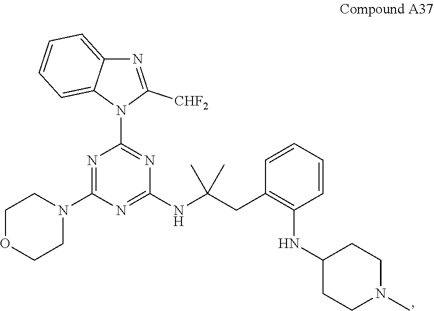 [SMILES: CN1CCC(Nc2ccccc2CC(C)(C)Nc2nc(N3CCOCC3)nc(-n3c(C(F)F)nc4ccccc43)n2)CC1]
or an isotopic variant thereof, a pharmaceutically acceptable salt, solvate, hydrate, or prodrug thereof.

74. The method of any one of claims 1-68, wherein the compound of Formula (I) is Compound A38: 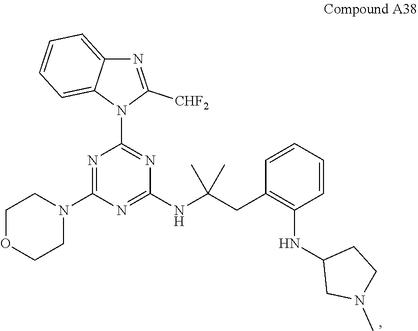 [SMILES: CN1CCC(Nc2ccccc2CC(C)(C)Nc2nc(N3CCOCC3)nc(-n3c(C(F)F)nc4ccccc43)n2)C1]
or an isotopic variant thereof, a pharmaceutically acceptable salt, solvate, hydrate, or prodrug thereof.

75. The method of any one of claims 1-68, wherein the compound of Formula (I) is Compound A41: 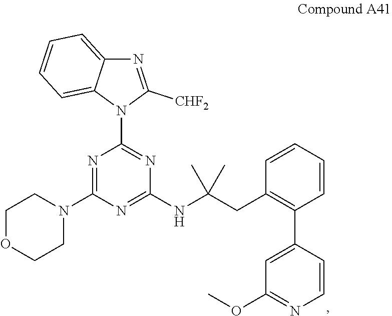 [SMILES: COc1cc(-c2ccccc2CC(C)(C)Nc2nc(N3CCOCC3)nc(-n3c(C(F)F)nc4ccccc43)n2)ccn1]
or an isotopic variant thereof, a pharmaceutically acceptable salt, solvate, hydrate, or prodrug thereof.

76. The method of any one of claims 1-68, wherein the compound of Formula (I) is Compound A42: 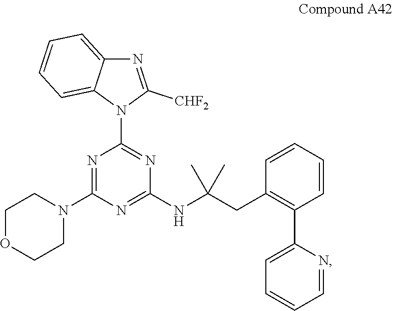 [SMILES: CC(C)(Cc1ccccc1-c1ccccn1)Nc1nc(N2CCOCC2)nc(-n2c(C(F)F)nc3ccccc32)n1]
or an isotopic variant thereof, a pharmaceutically acceptable salt, solvate, hydrate, or prodrug thereof.

77. The method of any one of claims 1-68, wherein the compound of Formula (I) is Compound A43: 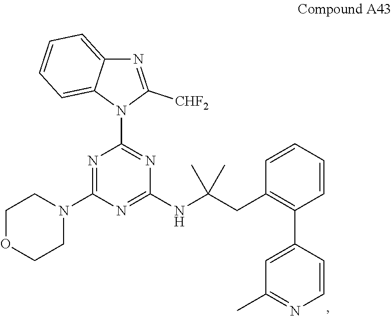 [SMILES: Cc1cc(-c2ccccc2CC(C)(C)Nc2nc(N3CCOCC3)nc(-n3c(C(F)F)nc4ccccc43)n2)ccn1]
or an isotopic variant thereof, a pharmaceutically acceptable salt, solvate, hydrate, or prodrug thereof.

78. The method of any one of claims 1-68, wherein the compound of Formula (I) is Compound A44: 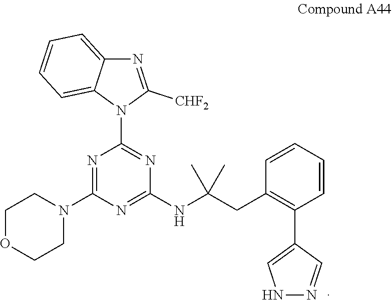 [SMILES: CC(C)(Cc1ccccc1-c1cn[nH]c1)Nc1nc(N2CCOCC2)nc(-n2c(C(F)F)nc3ccccc32)n1]
or an isotopic variant thereof, a pharmaceutically acceptable salt, solvate, hydrate, or prodrug thereof.

79. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject orally.

80. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule.
